(19) United States
(12) Patent Application Publication
(10) Pub. No.: US 2026/0253746 A1
(43) Pub. Date: Aug. 27, 2026

(54) SYSTEM AND METHOD FOR PREDICTING OR CHARACTERIZING IMMUNE RESPONSES

(71) Applicant: JANSSEN PHARMACEUTICA NV, Beerse (BE)

(72) Inventors: Joris VANDENBOSSCHE, Beerse (BE); Juan Jose PEREZ RUIXHO; Jose Ignacio FERNANDEZ DE TROCONIZ FERNANDEZ, Navarra (ES)

(73) Assignee: JANSSEN PHARMACEUTICA NV, Beerse (BE)

(21) Appl. No.: 18/704,208

(22) PCT Filed: Oct. 29, 2021
(86) PCT No.: PCT/IB2021/000756

§ 371 Date: Apr. 24, 2024

(51) Int. Cl. G16H 50/50; G16B 5/30; G16B 40/20; G16H 50/30

(52) U.S. Cl. CPC G16H 50/50; G16B 5/30; G16B 40/20; G16H 50/30

(57) ABSTRACT

Disclosed herein are methods for characterizing one or more immune responses of an immune system against a hepatitis B virus (HBV) infection, including: obtaining input values for a plurality of parameters associated with the HBV infection for simulation; generating a plurality of predicted quantitative values of the immune responses across one or more anatomical compartments by applying a quantitative system pharmacology (QSP) model to the input values of the plurality of parameters; and characterizing the immune responses of the immune system based on the plurality of predicted quantitative values as leading to one of a self-resolution of the HBV infection, continuation of an acute status of the HBV infection, or evolution to a chronic status of the HBV infection.

### RELATED APPLICATION

This application is a national stage application under 35 U.S.C. § 371 of International Patent Application No. PCT/IB2021/000756, filed on Oct. 29, 2021, which is incorporated herein by reference in its entirety for all purposes.

### BACKGROUND

Hepatitis B virus (HBV) infection can lead to acute HBV infection or to chronic HBV infection. In the vast majority (>95%) of adult exposures, the infected individuals are capable of mounting an effective immune response leading to infection resolution. Despite the large number of treatments for HBV, complete eradication of the virus from the system (i.e., virologic cure) is currently unattainable. Mathematical models have been developed to understand quantitatively the interplay between viral dynamics, including HBV, and the immune response. However, these models focus on certain aspects of the immune response in isolation or oversimplify the immune components of the immune system, limiting their utility to explore the role of each component of the immune system in the final response.

Therefore, there is a need for a model to adequately characterize and understand the biological processes that may lead e.g., to an acute or chronic status of the disease.

### SUMMARY

Embodiments of the invention disclosed herein involve implementing models for characterizing viral dynamics and components of the innate, adaptive, and tolerant immune response of multiple compartments and to predict immune responses of an immune system.

The systems and methods as described herein integrate information from multiple sources (e.g., in vitro data, clinical knowledge, as well as existing models) and across different organization levels (i.e., molecular, cellular, and organ), as well as clinical data from acute patients reported in the literature, to provide, as input, to a quantitative system pharmacology (QSP) model that describes the chronology and/or plausibility of an HBV-triggered immune response. The QSP model analyzes the relevance of the different immune pathways and biological processes, innate response and the cellular response on viral clearance. For example, moderate reductions of the proliferation of activated cytotoxic CD8+ lymphocytes or increased immunoregulatory effects can drive the system towards chronicity. From a quantitative perspective, the QSP model as described herein represents a valuable tool to understand the key processes involved in acute hepatitis B virus response, identify knowledge gaps, or evaluate pharmacologic targets.

As described herein, a quantitative system pharmacology (QSP) model is developed based on a topological representation characterizing the known interactions between the key elements of the HBV and the immune system, in terms of location, causality, and the nature of the relationship. Using the topological representation as the starting point, the multiscale QSP model characterizes mechanistically the dynamics and role of the different components of the immune system at a cellular level during an acute response against HBV, the potential drivers of HB chronicity. The QSP model can be used as a platform to evaluate pharmacologic targets.

Disclosed herein is a method for characterizing one or more immune responses of an immune system against a hepatitis B virus (HBV) infection, comprising: obtaining input values for a plurality of parameters associated with the HBV infection for simulation; generating a plurality of predicted quantitative values of the immune responses across one or more anatomical compartments by applying a quantitative system pharmacology (QSP) model to the input values of the plurality of parameters; and characterizing the immune responses of the immune system based on the plurality of predicted quantitative values as leading to one of a self-resolution of an acute HBV infection, continuation of an acute status of the HBV infection, or evolution to a chronic status of the HBV infection.

In various embodiments, the plurality of parameters comprise one or both of CTL proliferation rate constant (kprol_CTL) and HBV synthesis constant (ksyn_HBV).

In various embodiments, the plurality of parameters comprise one or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise five or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise ten or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise each of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise at least one of a first set of parameters and a second set of parameters, wherein the first set of parameters reflects physiological conditions, and wherein the second set of parameters describes a rate of different biological and disease processes.

In various embodiments, the one or more anatomical compartments comprise at least one of liver, plasma, bone marrow, lymphoid tissue, and lymph node (LN).

In various embodiments, the method further comprises characterizing an interaction between HBV and components of immune systems across the one or more anatomical compartments using the plurality of predicted quantitative values.

In various embodiments, the immune responses comprise at least one of innate immune response, adaptive immune response, and immunotolerant response, wherein the adaptive immune response comprises at least one of HBV-specific cellular adaptive response and HBV-specific humoral response.

In various embodiments, the immune system is initiated with a viral load arriving at a liver.

In various embodiments, generating a plurality of predicted quantitative values of the immune responses across one or more anatomical compartments comprises: simulating dynamics of the plurality of parameters using software, wherein the software comprises the Simbiology® toolbox from Matlab® (R2019a).

Additionally disclosed herein is a method for developing a quantitative system pharmacology (QSP) model to characterize one or more immune responses of an immune system against a hepatitis B virus (HBV) infection, the method comprising: building a QSP model comprising a plurality of parameters associated with the HBV infection; obtaining input values for the plurality of parameters for simulation; generating a plurality of predicted quantitative values of the immune responses across one or more anatomical compartments by applying the QSP model to the input values of the plurality of parameters for simulation; evaluating the QSP model by comparing the plurality of predicted quantitative values of the immune responses to observed data; assessing the QSP model by conducting an analysis; and analyzing behaviors predicted from the QSP model, wherein the immune responses of the immune system leads to one of self-resolution of an acute HBV infection, continuation of an acute status of the HBV infection, or evolution to a chronic status of the HBV infection.

In various embodiments, the plurality of parameters comprise one or both of CTL proliferation rate constant (kprol_CTL) and HBV synthesis constant (ksyn_HBV).

In various embodiments, the plurality of parameters comprise one or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise five or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise ten or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise each of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise at least one of a first set of parameters and a second set of parameters, wherein the first set of parameters reflects physiological conditions, and wherein the second set of parameters describes a rate of different biological and disease processes.

In various embodiments, the one or more anatomical compartments comprise at least one of liver, plasma, and lymph node (LN).

In various embodiments, the method further comprises characterizing interaction between HBV and key components of immune systems across the one or more anatomical compartments.

In various embodiments, the immune responses comprise at least one of innate immune response, adaptive immune response, and immunotolerant response, and wherein the adaptive immune response comprises at least one of HBV-specific cellular adaptive response and HBV-specific humoral response.

In various embodiments, the immune system is initiated with a viral load arriving at a liver.

In various embodiments, generating a plurality of predicted quantitative values of the immune responses across one or more anatomical compartments comprises: simulating dynamics of the plurality of parameters using software, wherein the software comprises the Simbiology® toolbox from Matlab® (R2019a).

In various embodiments, building a QSP model comprises: providing initial conditions and initial parameters for model entities and parameter estimates; implementing a plurality of biological entities across the one or more anatomical compartments; and implementing a plurality of biological processes, the biological processes comprising at least one of synthesis, degradation, and distribution through at least one of zero-, first-, and second-order rate constants.

In various embodiments, comparing the plurality of predicted quantitative values of the immune responses to observed data comprises at least one of: comparing a reproduction capability of the QSP model to general disease progression knowledge; and comparing typical model predictions to clinical data.

In various embodiments, assessing the QSP model by conducting an analysis comprises conducting at least one of a local sensitivity analysis or a parameter scan.

In various embodiments, analyzing behaviors predicted from the QSP model comprise evaluating relative contribution of at least one of the immune responses on a time profile of at least one relevant disease biomarker.

In various embodiments, the at least one relevant disease biomarker comprises at least one of viral load, HBVs antigens, IFNα, and alanine aminotransferase (ALT).

In various embodiments, analyzing behaviors predicted from the QSP model comprise evaluating a capability of the QSP model to predict development of chronicity of the immune system.

In various embodiments, the QSP model is based on a topological network.

In various embodiments, the topological network comprises a proposed interaction between HBV and the immune responses across the one or more anatomical compartments.

In various embodiments, the plurality of predicted quantitative values of the immune responses comprise time profiles for the plurality of parameters.

Additionally disclosed herein is a non-transitory computer readable medium for characterizing one or more immune responses of an immune system against a hepatitis B virus (HBV) infection, comprising instructions that, when executed by a processor, cause the processor to: obtain input values for a plurality of parameters associated with the HBV infection for simulation; generate a plurality of predicted quantitative values of the immune responses across one or more anatomical compartments by applying a quantitative system pharmacology (QSP) model to the input values of the plurality of parameters; and characterize the immune responses of the immune system based on the plurality of predicted quantitative values as leading to one of a self-resolution of an acute HBV infection, continuation of an acute status of the HBV infection, or evolution to a chronic status of the HBV infection.

In various embodiments, the plurality of parameters comprise one or both of CTL proliferation rate constant (kprol_CTL) and HBV synthesis constant (ksyn_HBV).

In various embodiments, the plurality of parameters comprise one or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise five or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise ten or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise each of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise at least one of a first set of parameters and a second set of parameters, wherein the first set of parameters reflects physiological conditions, and wherein the second set of parameters describes a rate of different biological and disease processes.

In various embodiments, the one or more anatomical compartments comprise at least one of liver, plasma, bone marrow, lymphoid tissue, and lymph node (LN).

In various embodiments, the non-transitory computer readable medium further comprises instructions that, when executed by the processor, cause the processor to characterize an interaction between HBV and components of immune systems across the one or more anatomical compartments using the plurality of predicted quantitative values.

In various embodiments, the immune responses comprise at least one of innate immune response, adaptive immune response, and immunotolerant response, wherein the adaptive immune response comprises at least one of HBV-specific cellular adaptive response and HBV-specific humoral response.

In various embodiments, the immune system is initiated with a viral load arriving at a liver.

In various embodiments, the instructions that cause the processor to generate a plurality of predicted quantitative values of the immune responses across one or more anatomical compartments comprises instructions that, when executed by the processor, cause the processor to: simulate dynamics of the plurality of parameters using software, wherein the software comprises the Simbiology® toolbox from Matlab® (R2019a).

Additionally disclosed herein is a non-transitory computer readable medium for developing a quantitative system pharmacology (QSP) model to characterize one or more immune responses of an immune system against a hepatitis B virus (HBV) infection, comprising instructions that, when executed by a processor, cause the processor to: build a QSP model comprising a plurality of parameters associated with the HBV infection; obtain input values for the plurality of parameters for simulation; generate a plurality of predicted quantitative values of the immune responses across one or more anatomical compartments by applying the QSP model to the input values of the plurality of parameters for simulation; evaluate the QSP model by comparing the plurality of predicted quantitative values of the immune responses to observed data; assess the QSP model by conducting an analysis; and analyze behaviors predicted from the QSP model, wherein the immune responses of the immune system leads to one of self-resolution of an acute HBV infection, continuation of an acute status of the HBV infection, or evolution to a chronic status of the HBV infection.

In various embodiments, the plurality of parameters comprise one or both of CTL proliferation rate constant (kprol_CTL) and HBV synthesis constant (ksyn_HBV).

In various embodiments, the plurality of parameters comprise one or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise five or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise ten or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise each of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise at least one of a first set of parameters and a second set of parameters, wherein the first set of parameters reflects physiological conditions, and wherein the second set of parameters describes a rate of different biological and disease processes.

In various embodiments, the one or more anatomical compartments comprise at least one of liver, plasma, and lymph node (LN).

In various embodiments, the non-transitory computer readable medium further comprises instructions that, when executed by the processor, cause the processor to characterize interaction between HBV and key components of immune systems across the one or more anatomical compartments.

In various embodiments, the immune responses comprise at least one of innate immune response, adaptive immune response, and immunotolerant response, and wherein the adaptive immune response comprises at least one of HBV-specific cellular adaptive response and HBV-specific humoral response.

In various embodiments, the immune system is initiated with a viral load arriving at a liver.

In various embodiments, the instructions that cause the processor to generate a plurality of predicted quantitative values of the immune responses across one or more anatomical compartments comprises instructions that, when executed by the processor, cause the processor to: simulate dynamics of the plurality of parameters using software, wherein the software comprises the Simbiology® toolbox from Matlab® (R2019a).

In various embodiments, the instructions that cause the processor to build a QSP model comprises instructions that, when executed by the processor, cause the processor to: provide initial conditions and initial parameters for model entities and parameter estimates; implement a plurality of biological entities across the one or more anatomical compartments; and implement a plurality of biological processes, the biological processes comprising at least one of synthesis, degradation, and distribution through at least one of zero-, first-, and second-order rate constants.

In various embodiments, the instructions that cause the processor to compare the plurality of predicted quantitative values of the immune responses to observed data comprises instructions that, when executed by the processor, cause the processor to: compare a reproduction capability of the QSP model to general disease progression knowledge; or compare typical model predictions to clinical data.

In various embodiments, the instructions that cause the processor to assess the QSP model by conducting an analysis comprises instructions that, when executed by the processor, cause the processor to conduct at least one of a local sensitivity analysis or a parameter scan.

In various embodiments, the instructions that cause the processor to analyze behaviors predicted from the QSP model comprise instructions that, when executed by the processor, cause the processor to evaluate relative contribution of at least one of the immune responses on a time profile of at least one relevant disease biomarker.

In various embodiments, the at least one relevant disease biomarker comprises at least one of viral load, HBVs antigens, IFNα, and alanine aminotransferase (ALT).

In various embodiments, the instructions that cause the processor to analyze behaviors predicted from the QSP model comprise instructions that, when executed by the processor, cause the processor to evaluate a capability of the QSP model to predict development of chronicity of the c system.

In various embodiments, the QSP model is based on a topological network.

In various embodiments, the topological network comprises a proposed interaction between HBV and the immune responses across the one or more anatomical compartments.

In various embodiments, the plurality of predicted quantitative values of the immune responses comprise time profiles for the plurality of parameters.

Additionally disclosed herein is a system for characterizing one or more immune responses of an immune system against a hepatitis B virus (HBV) infection, comprising: a parameter value module configured to obtain input values for a plurality of parameters associated with the HBV infection for simulation; a model deployment module configured to generate a plurality of predicted quantitative values of the immune responses across one or more anatomical compartments by applying a quantitative system pharmacology (QSP) model to the input values of the plurality of parameters; and a response characterization module configured to characterize the immune responses of the immune system based on the plurality of predicted quantitative values as leading to one of a self-resolution of an acute HBV infection, continuation of an acute status of the HBV infection, or evolution to a chronic status of the HBV infection.

In various embodiments, the plurality of parameters comprise one or both of CTL proliferation rate constant (kprol_CTL) and HBV synthesis constant (ksyn_HBV).

In various embodiments, the plurality of parameters comprise one or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise five or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise ten or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise each of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise at least one of a first set of parameters and a second set of parameters, wherein the first set of parameters reflects physiological conditions, and wherein the second set of parameters describes a rate of different biological and disease processes.

In various embodiments, the one or more anatomical compartments comprise at least one of liver, plasma, bone marrow, lymphoid tissue, and lymph node (LN).

In various embodiments, the computer system further characterizes an interaction between HBV and components of immune systems across the one or more anatomical compartments using the plurality of predicted quantitative values.

In various embodiments, the immune responses comprise at least one of innate immune response, adaptive immune response, and immunotolerant response, wherein the adaptive immune response comprises at least one of HBV-specific cellular adaptive response and HBV-specific humoral response.

In various embodiments, the immune system is initiated with a viral load arriving at a liver.

In various embodiments, the prediction engine generates the plurality of predicted quantitative values of the immune responses across one or more anatomical compartments by simulating dynamics of the plurality of parameters using software, wherein the software comprises the Simbiology® toolbox from Matlab® (R2019a).

Additionally disclosed herein is a system for developing a quantitative system pharmacology (QSP) model to characterize one or more immune responses of an immune system against a hepatitis B virus (HBV) infection, comprising: a model building module configured to build a QSP model comprising a plurality of parameters associated with the HBV infection, wherein the model building module comprises: an input engine configured to obtain input values for the plurality of parameters for simulation; a prediction engine configured to generate a plurality of predicted quantitative values of the immune responses across one or more anatomical compartments by applying the QSP model to the input values of the plurality of parameters for simulation; and an evaluation engine configured to evaluate the QSP model by comparing the plurality of predicted quantitative values of the immune responses to observed data, assess the QSP model by conducting an analysis, and analyze behaviors predicted from the QSP model, wherein the immune responses of the immune system leads to one of self-resolution of an acute HBV infection, continuation of an acute status of the HBV infection, or evolution to a chronic status of the HBV infection.

In various embodiments, the plurality of parameters comprise one or both of CTL proliferation rate constant (kprol_CTL) and HBV synthesis constant (ksyn_HBV).

In various embodiments, the plurality of parameters comprise one or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise five or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise ten or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise each of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.

In various embodiments, the plurality of parameters comprise at least one of a first set of parameters and a second set of parameters, wherein the first set of parameters reflects physiological conditions, and wherein the second set of parameters describes a rate of different biological and disease processes.

In various embodiments, the one or more anatomical compartments comprise at least one of liver, plasma, and lymph node (LN).

In various embodiments, the model building module further comprises a characterization engine configured to characterize interaction between HBV and key components of immune systems across the one or more anatomical compartments.

In various embodiments, the immune responses comprise at least one of innate immune response, adaptive immune response, and immunotolerant response, and wherein the adaptive immune response comprises at least one of HBV-specific cellular adaptive response and HBV-specific humoral response.

In various embodiments, the immune system is initiated with a viral load arriving at a liver.

In various embodiments, generate a plurality of predicted quantitative values of the immune responses across one or more anatomical compartments comprises: simulating dynamics of the plurality of parameters using software, wherein the software comprises the Simbiology® toolbox from Matlab® (R2019a).

In various embodiments, build a QSP model comprises: providing initial conditions and initial parameters for model entities and parameter estimates to the model building module, wherein the model building module implements a plurality of biological entities across the one or more anatomical compartments, and wherein the model building module implements a plurality of biological processes, the biological processes comprising at least one of synthesis, degradation, and distribution through at least one of zero-, first-, and second-order rate constants.

In various embodiments, comparing the plurality of predicted quantitative values of the immune responses to observed data comprises at least one of: comparing a reproduction capability of the QSP model to general disease progression knowledge using the evaluation engine; and comparing typical model predictions to clinical data using the evaluation engine.

In various embodiments, assess the QSP model by conducting an analysis comprises conducting at least one of a local sensitivity analysis or a parameter scan.

In various embodiments, analyze behaviors predicted from the QSP model comprise evaluating relative contribution of at least one of the immune responses on a time profile of at least one relevant disease biomarker using the evaluation engine.

In various embodiments, the at least one relevant disease biomarker comprises at least one of viral load, HBVs antigens, IFNα, and alanine aminotransferase (ALT).

In various embodiments, analyze behaviors predicted from the QSP model comprise evaluating a capability of the QSP model to predict development of chronicity of the immune system.

In various embodiments, the QSP model is based on a topological network.

In various embodiments, the topological network comprises a proposed interaction between HBV and the immune responses across the one or more anatomical compartments.

In various embodiments, the plurality of predicted quantitative values of the immune responses comprise time profiles for the plurality of parameters.

### BRIEF DESCRIPTION OF THE DRAWINGS

These and other features, aspects, and advantages of the present invention will become better understood with regard to the following description and accompanying drawings. It is noted that wherever practicable similar or like reference numbers may be used in the figures and may indicate similar or like functionality. For example, a letter after a reference numeral, such as “compartment value 240A,” indicates that the text refers specifically to the element having that particular reference numeral. A reference numeral in the text without a following letter, such as “compartment value 240,” refers to any or all of the elements in the figures bearing that reference numeral (e.g. “compartment value 240” in the text refers to reference numerals “compartment value 240A,” “compartment value 240B,” “compartment value 240C,” etc in the figures).

FIG. 1A illustrates a system environment overview for predicting or characterizing immune responses against an hepatitis B virus infection, in accordance with an embodiment.

FIG. 1B illustrates a block diagram of the hepatitis B virus response system, in accordance with an embodiment.

FIG. 2 illustrates a block diagram for predicting immune responses against hepatitis B virus, in accordance with an embodiment.

FIG. 3 illustrates a flow process for characterizing immune responses against hepatitis B virus, in accordance with an embodiment.

FIG. 4 illustrates an example computer for implementing the entities shown in FIGS. 1A, 1, 2, and 3.

FIG. 5 illustrates example development of a QSP model.

FIG. 6 illustrates a schematic representation of the QSP model.

FIG. 7 illustrates disease course of acute hepatitis B (AHB).

FIG. 8 illustrates evaluation of the final QSP model associated with AHB.

FIG. 9 illustrates a local sensitivity analysis.

FIG. 10A illustrates impact of 50% change on peak HBV DNA levels or time to cure when varying one parameter at a time.

FIG. 10B illustrates impact of varying CTL proliferation rate constant (kprol_CTL) or HBV synthesis constant (ksyn_HBV) on the time course of circulating DNA viral levels (HBV DNA).

FIG. 11A illustrates impact of 50% change on peak HBV DNA levels or time to cure (defined as HBV DNA <20 IU/L & maximum simulation time of 45 weeks) when varying one parameter at a time.

FIG. 11B illustrates impact of varying CTL proliferation rate constant (k_{prol_CTL}) or HBV synthesis constant (k_{syn_HBV}) on the time course of circulating DNA viral levels (HBV DNA).

FIG. 12 illustrates a knock-out analysis based on model predicted time course of viral load (HBV DNA), surface hepatitis B antigen (HBsAg), and alanine aminotransferase (ALT) in blood under different knock-out scenarios: no perturbation (reference), no NK activation (k_{act_NK}=0), no CTL activation (k_{act_CTL}=0), no Bcell activation (k_{act_Bcell}==0), no DC activation (k_{act_DC}=0), increased Treg activation (CTL_{50_Treg_prol}=200000) or increased Treg sensitivity to CTL exhaustion (Treg_{50_CTL_exh}=200000).

FIG. 13 illustrates predicted time profiles for the different model entities across three 3 compartments including blood, liver and lymph.

### DETAILED DESCRIPTION

### I. Definitions

Terms used in the embodiments and specification are defined as set forth below unless otherwise specified.

The terms “subject” or “patient” are used interchangeably and encompass a cell, tissue, or organism, human or non-human, whether in vivo, ex vivo, or in vitro, male or female.

The term “obtaining input values for a plurality of parameters” encompasses obtaining one or more parameters from an external (e.g., publicly available) database or obtaining one or more parameters from a locally available data store. Obtaining input values for one or more parameters can encompass performing steps of pulling (or capturing) the one or more parameters from the external (e.g., publicly available) database or the locally available data store. The phrase can also encompass receiving input values for one or more parameters, e.g., from a party that has performed the steps of obtaining the input values for one or more parameters from the external (e.g., publicly available) database or the locally available data store. Input values for the one or more parameters can be obtained by one of skill in the art via a variety of known ways including stored on a storage memory. In various embodiments, obtaining input values for one or more parameters can encompass obtaining input values for one or more parameters for a particular subject. Thus, the input values for one or more parameters can be subject-specific.

The term “QSP model” and “quantitative systems pharmacology model” refer to a quantitative model that integrates biological processes triggered by an infection (e.g., upon AHB infection) in a quantitative framework such as a topological network. A developed QSP model may be successfully applied to clinical data.

The term “topological network” refers to a network that predicts the interaction between the virus and key players of the innate, adaptive, and immunoregulatory system across relevant compartments such as liver (LV), plasma (PL) and lymph node (LN).

Abbreviations used herein are defined as below: AHB: acute hepatitis B; ALT: alanine aminotransferase; anti-HBc: specific antibodies against core hepatitis B antigen; anti-HBs: specific antibodies against surface hepatitis B antigen; HBsAg: hepatitis B surface antigen, CHB: chronic hepatitis B; CTL: antigen-specific cytotoxic T lymphocytes; CTL*: activated CTL; CTLm: memory CTL; DC: dendritic cells; dHep: debris hepatocytes; HBV: hepatitis B virus, HBV DNA: circulating DNA levels of HBV; Hep: hepatocytes; Heptot: total hepatocytes; iHep: infected hepatocytes; IFN: interferon; LN: lymph node; LPC: long-lived plasma cell; LV: liver; NK: natural killer; NK*: activated NK; ODE: ordinary differential equations; PB: plasmablast; PC: plasma cell; pDC: plasmacytoid DC; PL: plasma; QSP: quantitative systems pharmacology; TRAIL: tumor necrosis factor-related apoptosis-inducing ligand; Treg: regulatory T cells.

It must be noted that, as used in the specification, the singular forms “a,” “an” and “the” include plural referents unless the context clearly dictates otherwise.

### II. System Environment Overview

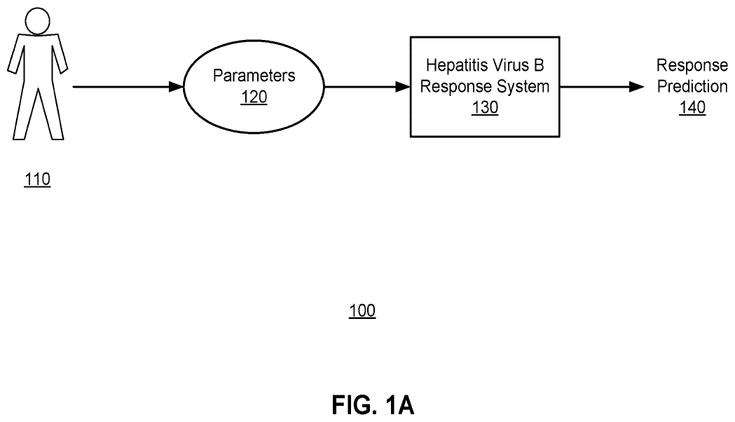

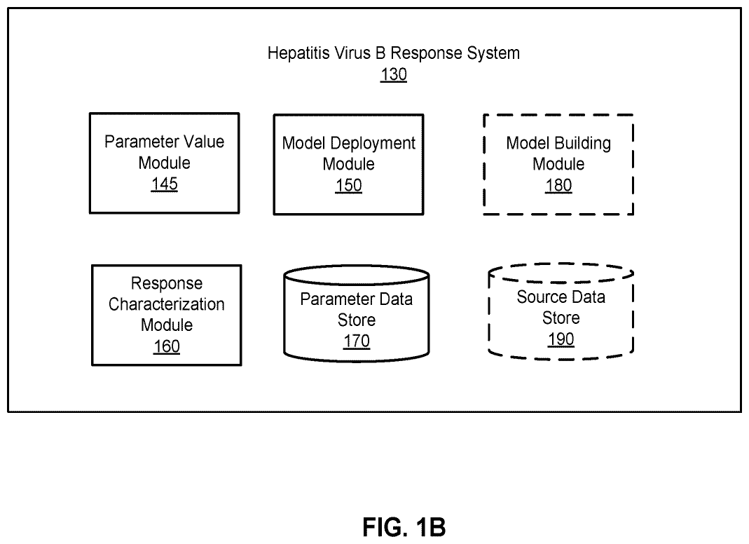

Figure (FIG. 1A depicts a system environment overview 100 for predicting, capturing, or characterizing immune responses against an HBV infection, in accordance with an embodiment. The system environment 100 provides context in order to introduce a subject (or patient) 110, and a hepatitis B virus response system 130 for generating one or more response predictions 140.

The system environment 100 may include one or more subjects 110 who were enrolled in a study conducted by the hepatitis B virus response system 130. In various embodiments, the subject 110 may have met eligibility criteria for enrollment in the study. For example, the subject 110 may have been previously diagnosed with a hepatitis B As another example, the subject 110 may have been enrolled in a clinical trial that tested a therapeutic intervention for treating the hepatitis B. Although FIG. 1A depicts one subject 110, in various embodiments, the system environment overview 100 may include two or more subjects 110 that were enrolled in a study conducted by the hepatitis B virus response system 130.

In various embodiments, the system environment 100 need not include subjects 110 and instead, parameters 120 can be merely included in the system environment 100 for simulation purposes. For example, the parameters can be varied or tweaked for enabling simulations of immune responses to hepatitis B based on the varied or tweaked parameters.

The hepatitis B virus response system 130 obtains and analyzes the input values of a plurality of parameters 120 associated with the HBV infection describing physiological conditions (e.g., organ volumes or entity levels at baseline) and the rate of the different biological and disease processes, and generates a response prediction 140 by applying a QSP model (e.g., based on a topological network). In various embodiments, the hepatitis B virus response system 130 is a party or is operated by a party.

In various embodiments, the hepatitis B virus response system 130 applies a QSP model to analyze or evaluate the plurality of parameters 120 associated with HBV infection to generate a response prediction 140.

In various embodiments, the plurality of parameters 120 include a set of parameters that reflect physiological conditions. In various embodiments, the plurality of parameters 120 include a set of parameters that describe rates of different biological and disease processes. In various embodiments, the plurality of parameters 120 include both a set of parameters that reflect physiological conditions and a set of parameters that describe a rate of different biological and disease processes.

In various embodiments, the plurality of parameters 120 include one or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg. Further description and example parameters are described in Table S1. In various embodiments, the plurality of parameters 120 include five or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg. In various embodiments, the plurality of parameters 120 include ten or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg. In various embodiments, the plurality of parameters 120 include each of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg. In particular embodiments, the plurality of parameters 120 includes one of CTL proliferation rate constant (kprol_CTL) and HBV synthesis constant (ksyn_HBV). In particular embodiments, the plurality of parameters includes both CTL proliferation rate constant (kprol_CTL) and HBV synthesis constant (ksyn_HBV).

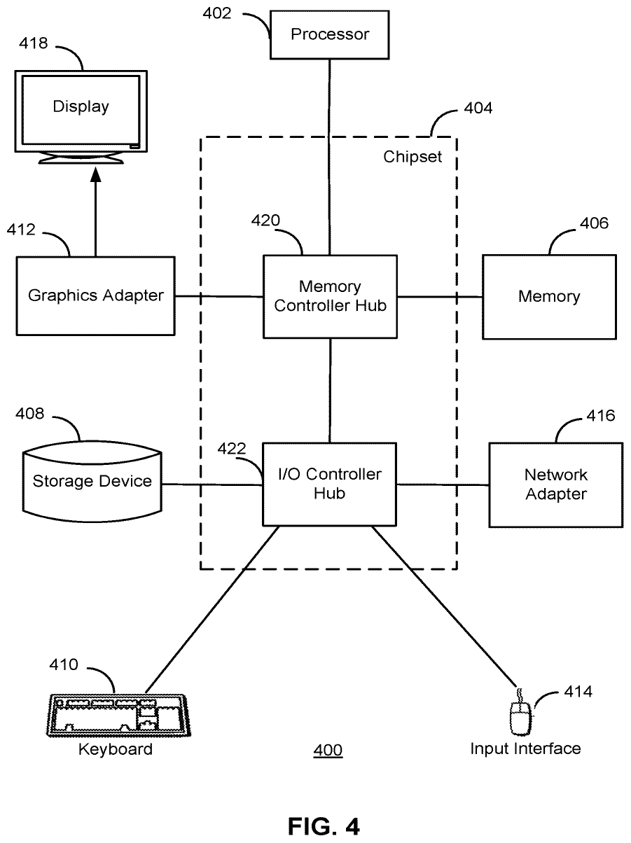

In various embodiments, the hepatitis B virus response system 130 can include one or more computers, embodied as a computer system 400 as discussed below with respect to FIG. 4. Therefore, in various embodiments, the steps described in reference to the hepatitis B virus response system 130 are performed in silico.

The response prediction 140 is generated by the hepatitis B virus response system 130 and includes immune response of an immune system (e. g., an immune system of a subject 110) against an HBV infection. In various embodiments, the immune responses include at least one of innate immune response, adaptive immune response, and/or immunotolerant response. In various embodiments, the immune responses include each of innate immune response, adaptive immune response, and/or immunotolerant response. In various embodiments, the adaptive immune response includes HBV-specific cellular adaptive response and/or HBV-specific humoral response.

Reference is now made to FIG. 1B which depicts a block diagram illustrating the computer logic components of the hepatitis B virus response system 130, in accordance with an embodiment. The components of the hepatitis B virus response system 130 are hereafter described in reference to two phases: 1) a development phase and 2) a deployment phase. More specifically, the development phase refers to the building, developing, and/or evaluating of a QSP model using source (or training) data (e.g. known or observed data captured from one or more sources such as clinical trials, literature, publication, etc). The simulated results (e.g., a response prediction) generated from the QSP model may have be known and/or may be unknown. Therefore, the QSP model is developed such that during the deployment phase, implementation of the QSP model enables the generation of a response prediction (e.g., response prediction 140 in FIG. 1A).

As shown in FIG. 1B, the hepatitis B virus response system 130 includes a parameter value module 145, a model deployment module 150, a response characterization module 160, and a parameter data store 170, a model building module 180, and a source data store 190. In various embodiments, the hepatitis B virus response system 130 can be configured differently with additional or fewer modules. For example, a hepatitis B virus response system 130 need not include the parameter data store 170. As another example, the hepatitis B virus response system 130 need not include the model building module 180 or the source data store 190 (as indicated by their dotted lines in FIG. 1B), and instead, the model building module 180 or the source data store 190 are employed by a different system and/or party.

Generally, the parameter value module 145 processes (e.g., extracts) input values of one or more parameters that may be obtained or stored in the parameter data store 170, and provides the input values to the model deployment module 150. The model deployment module 150 implements a QSP model to analyze features of the extracted parameter values associate with hepatitis B virus (e.g., hepatitis B virus in a subject 110 in FIG. 1A) to predict immune responses for an immune system (e.g., immune system of a subject 110). The response characterization module 160 generates predictions informative of the immune responses of the immune system.

The model deployment module 150 implements a QSP model to analyze one or more parameters saved in the parameter data store 170, and processed by the parameter value module 145 to generate a response prediction (e.g., response prediction 140 in FIG. 1A) for an immune system of a subject (e.g., subject 110 in FIG. 1A). In various embodiments, the QSP model is based on a topological network.

In various embodiments, the model deployment module 150 implements a QSP model for analyzing extracted values of parameters associated with HBV of an immune system, and generates a plurality of predicted quantitative values of the immune responses across one or more anatomical compartments. In various embodiments, the anatomical compartments include at least one of liver, plasma, bone marrow, lymph tissue (e.g., gut associated lymph tissue), and lymph node (LN). In various embodiments, the anatomical compartments include each of liver, plasma, bone marrow, lymph tissue (e.g., gut associated lymph tissue), and lymph node (LN). In various embodiments, the model deployment module 150 includes one or more prediction engines to generate a plurality of predicted quantitative values of the immune responses across one or more anatomical compartments.

The response characterization module 160 characterizes the immune responses of the immune system based on the plurality of predicted quantitative values as leading to one of self-resolution of an acute HBV infection, continuation of an acute status of the HBV infection, or evolution to a chronic status of the HBV infection. In various embodiments, the response characterization module 160 further characterizes an interaction between HBV and components of immune systems across the one or more anatomical compartments using the plurality of predicted quantitative values generated by the model deployment module 150. In various embodiments, the response characterization module 160 is or includes one or more response characterization engines to characterize the immune responses of the immune system based on the plurality of predicted quantitative values as leading to one of self-resolution of an acute HBV infection, continuation of an acute status of the HBV infection, or evolution to a chronic status of the HBV infection.

The model building module 180 builds or develops a QSP model for implementation (e.g., by the model deployment module 150) to predict or characterize the immune responses of the immune system leads to one of self-resolution of an acute HBV infection, continuation of an acute status of the HBV infection, or evolution to a chronic status of the HBV infection by using the source data, such as a plurality of parameters associated with the HBV infection, stored in the source data store 190. In various embodiments, the model building module 180 is configured to build the QSP model. In various embodiments, the model building module 180 includes an input engine configured to obtain input values for the plurality of parameters for simulation. In various embodiments, the model building module 180 includes a prediction engine configured to generate a plurality of predicted quantitative values of the immune responses across one or more anatomical compartments by applying the QSP model to the input values of the plurality of parameters for simulation. In various embodiments, the model building module 180 includes an evaluation engine configured to evaluate the QSP model by comparing the plurality of predicted quantitative values of the immune responses to observed data, assess the QSP model by conducting an analysis, and/or analyze behaviors predicted from the QSP model. In various embodiments, the model building module 180 includes a characterization engine configured to characterize interaction between HBV and key components of immune systems across the one or more anatomical compartments.

The parameter data store 170 stores parameters associated with hepatitis B virus (e.g., for subject 110 in FIG. 1A) for the parameter value module 145 to process. Example parameters are described in Table S1.

The source data store 190 stores data for the model building module 180 to develop the QSP model.

### III. Methods for Simulating and/or Generating Response Prediction

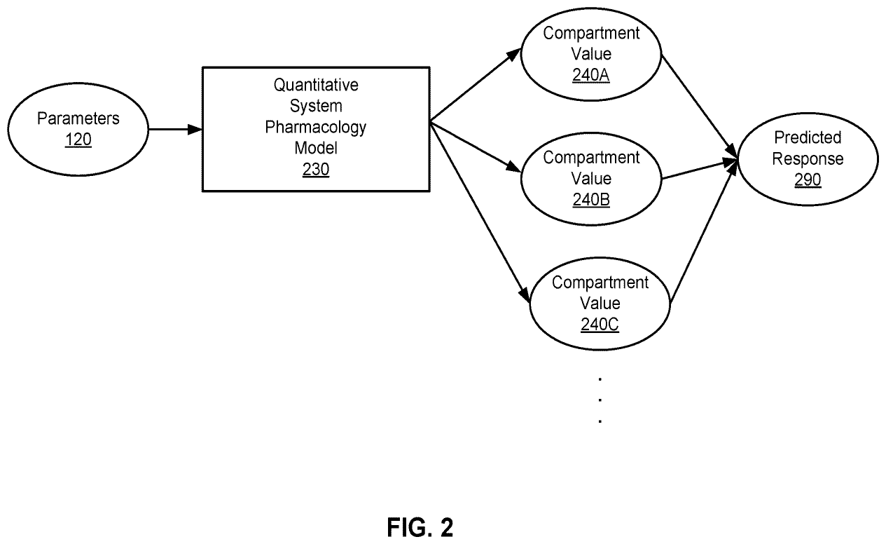

Embodiments described herein include methods for predicting immune responses of an immune system against a hepatitis B virus infection. Such methods can be performed by the hepatitis B virus response system 130, such as by the parameter value module 145, the model deployment module 150, and the response characterization module 160, as described in FIG. 1B. Reference will further be made to FIG. 2, which depicts an example flow diagram 200 for predicting the immune responses, in accordance with an embodiment. In various embodiments, methods for predicting immune responses (e.g., predicting immune responses not known or captured from source data) of an immune system against a hepatitis B virus infection are performed after characterizing or capturing immune responses (e.g., characterizing immune responses known or captured from source data) of an immune system against a hepatitis B virus infection.

As shown in FIG. 2, a QSP model 230 is implemented to generate a plurality of predicted quantitative values 240A, 240B, 240C, of the immune responses across one or more anatomical compartments based on input values of a plurality of parameters 120 (e.g., physiological parameters and/or rate parameters). In various embodiments, physiological parameters refer to parameters of constant value over time. In various embodiments, the physiological parameters may be the end result of a dynamic process (e.g., a biological and/or disease process). For example, the physiological parameters include liver volume, one or more rate (or ratio) constants, and/or other related parameters in Table S1. In various embodiments, the rate parameters describe dynamic processes, and thus can change values over time. In various embodiments, the rate parameters (e.g., rate parameters in Table S1) describe a rate (or ratio) of one or more biological and/or disease processes.

Generally, the plurality of predicted quantitative values 240A, 240B, 240C, of the immune responses across one or more anatomical compartments are further used to generate a predicted response 290 of the immune system. FIG. 2 depicts different compartments values 240 for different anatomical compartments. For example, compartment value 240A may correspond to a first anatomical compartment, compartment value 240B may correspond to a second anatomical compartment, and compartment value 240C may correspond to a third anatomical compartment, and so on. In various embodiments, the QSP model 230 may output multiple compartment values for each compartment. For example, there may be multiple compartment values 240A for a first anatomical compartment, multiple compartment values 240B for a second anatomical compartment, multiple compartment values 240C for a third anatomical compartment, and so on.

In various embodiments, there are at least five compartment values for a first anatomical compartment, at least five compartment values for a second anatomical compartment, and at least five compartment values for a third anatomical compartment. In various embodiments, there are at least ten compartment values for a first anatomical compartment, at least ten compartment values for a second anatomical compartment, and at least ten compartment values for a third anatomical compartment.

Generally, the parameters 120 (e.g., physiological parameters and/or rate parameters) are associated with an HBV infection. In various embodiments, the physiological parameters and/or rate parameters may be the parameters 120 or a subset of the parameters 120 described above in reference to FIG. 1A). In various embodiments, the physiological parameters and/or rate parameters may be obtained from one or more subjects (e.g., subjects 110 in FIG. 1A). In various embodiments, the physiological parameters and/or rate parameters are obtained from one or more data sources.

The QSP model 230 is applied to the input values of the plurality of parameters 120 using the model deployment module 150 (described in FIG. 1B) to generate a plurality of compartment values 240A, 240B, 240C representing immune responses of the compartments. In various embodiments, the QSP model 230 is previously developed and evaluated on source data (e.g., source data saved in source data store 190 in FIG. 1B). In various embodiments, a QSP model 230 is embodied as a set of equations that models the immune responses of the various compartments. An exemplary QSP model 230 is described below in Example section and Supplementary Appendix 1. Such an exemplary QSP model can account for a total of 32 biological entities across ≥1 compartments, described through 41 ordinary differential equations (ODEs) and 6 analytical equations.

In various embodiments, the plurality of compartment values 240A, 240B, 240C include predicted immune responses such as innate immune response, adaptive immune response, and/or immunotolerant response. In various embodiments, the adaptive immune response includes HBV-specific cellular adaptive response and/or HBV-specific humoral response. Methods for determining a site performance prediction 240 are described herein.

As shown in FIG. 2, the plurality of compartment values 240A, 240B, 240C are then used to character or predict predicted response 290 of an immune system. In various embodiments, the predicted response 290 includes time profiles for a plurality of model parameters (e.g., parameters 120 in FIG. 1A) across one or more anatomical compartments. In various embodiments, the anatomical compartments include at least one of liver, plasma, bone marrow, lymph tissue (e.g., gut associated lymph tissue), and lymph node (LN). In various embodiments, the anatomical compartments include each of liver, plasma, bone marrow, lymph tissue (e.g., gut associated lymph tissue), and lymph node (LN).

Generally, the plurality of compartment values 240A, 240B, 240C and/or the predicted response 290 are generated (e.g., calculated, simulated, etc.) by implementing one or more ordinary differential equations (ODEs) built in the QSP model. In various embodiments, the one or more ODEs are equations provided in the Supplementary Appendix 1. For example, equation 20 in the Supplementary Appendix 1 can lead to the calculation of the predicted response 290. As another example, equation 21 is used to further convert units of calculated results from equation 20.

In various embodiments, the predicted response 290 is any one of a self-resolution of an acute HBV infection, continuation of an acute status of the HBV infection, or evolution to a chronic status of the HBV infection. In various embodiments, the predicted response 290 is any one of a self-resolution of HBV infection, acute HBV infection, or chronic HBV infection. In various embodiments, the self-resolution of an acute HBV infection refers to a clearance of HBV and/or infected hepatocytes (iHep). In various embodiments, the self-resolution of an acute HBV infection as obtained by HBV plasma is determined if the DNA viral levels (HBV DNA) are less than 20, 25, 30, 35, or 40 IU/ml at X weeks, wherein X is any of 5 weeks, 10 weeks, 15 weeks, 20 weeks, or 25 weeks. In various embodiments, the self-resolution of an acute HBV infection as obtained by HBV plasma is determined if the DNA viral levels (HBV DNA) are at very low (e.g., undetectable) according to development guidelines. In various embodiments, the self-resolution of an acute HBV infection as obtained by HBV plasma is determined by the clearance or absence of surface hepatitis B antigen (HBsAg) in the periphery area.

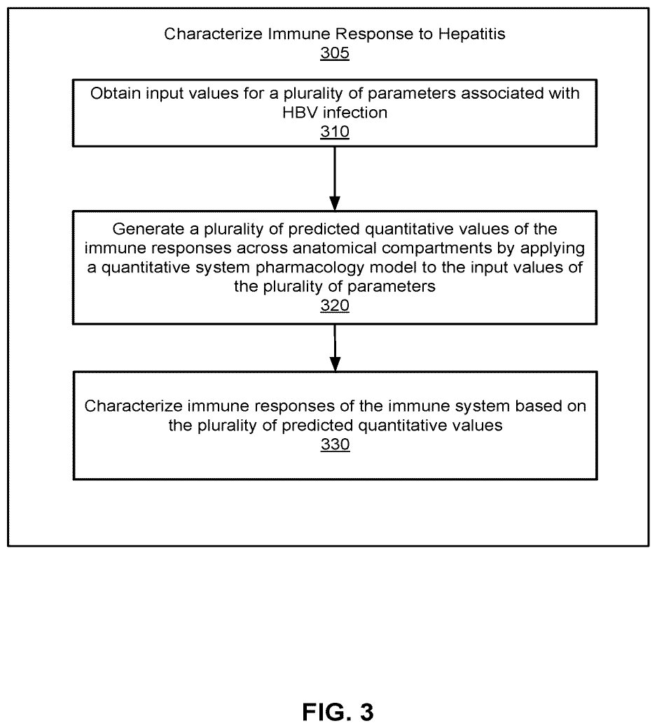

Reference is now made to FIG. 3, which depicts a flow diagram 305 for characterizing immune response against a HBV infection, in accordance with an embodiment.

At step 310, input values for a plurality of parameters associated with the HBV infection are obtained (e.g., using the parameter value module 145 in FIG. 1B). In various embodiments, input values for a plurality of parameters associated with the HBV infection for simulation are from one subject (e.g., subject 110 in FIG. 1A). In various embodiments, input values for a plurality of parameters associated with the HBV infection for simulation are from multiple subjects (e.g., subjects 110 in FIG. 1A). In various embodiments, the input values for a plurality of parameters associated with the HBV infection for simulation may be a statistical combination of values from multiple subjects. For example, the input values for a plurality of parameters may be an average, a median, or a mode value across values from multiple subjects. The obtained input values for a plurality of parameters associated with the HBV infection are provided, as input, to implement a QSP model (e.g., QSP model 230 in FIG. 2) for simulation.

At step 320, a plurality of predicted quantitative values of the immune responses across anatomical compartments are generated by applying a QSP model to the input values of the plurality of parameters. In various embodiments, generating a plurality of predicted quantitative values of the immune responses across one or more anatomical compartments further includes simulating dynamics of the plurality of parameters using software (e.g., the Simbiology® toolbox from Matlab® (R2019a)).

At step 330, immune responses of the immune system based on the plurality of predicted quantitative values are characterized, leading to a predicted response (e.g. predicted response 290 in FIG. 2) that is any one of self-resolution of an acute HBV infection, continuation of an acute status of the HBV infection, or evolution to a chronic status of the HBV infection. In various embodiments, the HBV may be self-resolving and therefore, no pharmacological intervention is needed. In various embodiments, further steps of pharmacological interventions (e.g., treatment, therapy, etc) may be performed based on the predicted response. Altogether, models in presently disclosed embodiments, which may start with an acute HBV infection, are beneficial for providing a trajectory of cure according to the predicted response.

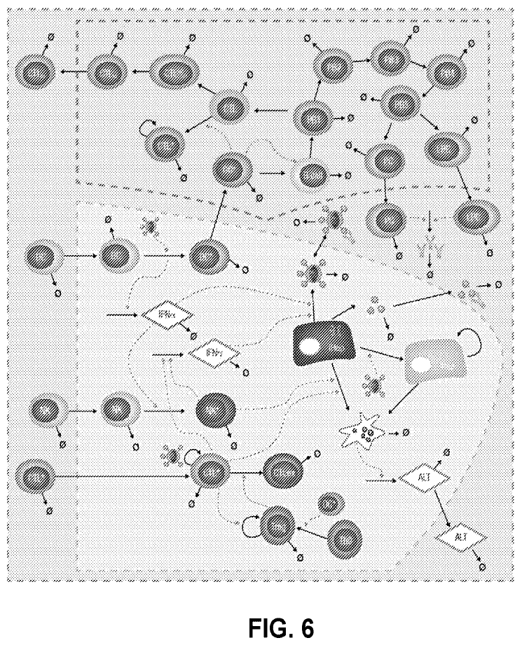

Referring again to FIGS. 2 and 3, the implementation of the QSP model can be used in developing phase, prior to the deployment phase, as described herein. In various embodiments, the developing and/or building of the QSP model is based on a topological network (e.g., topological network shown in FIG. 6) that includes a proposed interaction between HBV and the immune responses across the one or more anatomical compartments.

Generally, the building of the QSP model further includes: providing initial conditions and initial parameters for model entities and parameter estimates; implementing a plurality of biological entities across the one or more anatomical compartments; and/or implementing a plurality of biological processes, the biological processes comprising at least one of synthesis, degradation, and distribution through at least one of zero-, first-, and second-order rate constants. Furthermore, additional steps may be applied to build, evaluate, assess, and/or analyze the QSP model and/or responses predicted from the QSP model. For example, the QSP model may be evaluated by comparing the plurality of predicted quantitative values of the immune responses to observed data (e.g., by comparing a reproduction capability of the QSP model to general disease progression knowledge; and/or comparing typical model predictions to clinical data). As another example, the QSP model may be assessed by conducting an analysis (e.g., by conducting at least one of a local sensitivity analysis or a parameter scan). As another example, behaviors predicted from the QSP model may be analyzed, such as by evaluating relative contribution of at least one of the immune responses on a time profile of at least one relevant disease biomarker (e.g., viral load, HBVs antigens, IFNα, and alanine aminotransferase (ALT)), and/or by evaluating a capability of the QSP model to predict development of chronicity of the system.

### IV. Computer Implementation

The methods of the invention, including the methods of implementing a QSP model for predicting performance of clinical trials and/or pharmacological interventions s, are, in some embodiments, performed on one or more computers.

For example, the building and deployment of a QSP model can be implemented in hardware or software, or a combination of both. In one embodiment of the invention, a machine-readable storage medium is provided, the medium comprising a data storage material encoded with machine readable data which, when using a machine programmed with instructions for using said data, is capable of executing the training or deployment of QSP model and/or displaying any of the datasets or results described herein. The invention can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods of the invention, including the methods for predicting immune responses, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the embodiments, a “cloud-computing environment” is an environment in which cloud computing is employed.

In some embodiments, the methods of the invention, including the methods for characterizing or predicting immune responses, are performed on one or more computers in a grid computing system environment (e.g., in a parallel processing environment). In this description, “parallel computing” is defined as a model for enabling calculations and/or simulations of separate parts of an overall computing task simultaneously on multiple processors (e.g., multiple central processing units (CPUs)).

FIG. 4 illustrates an example computer for implementing the entities shown in FIGS. 1A, 1B, 2, and 3. The computer 400 includes at least one processor 402 coupled to a chipset 404. The chipset 404 includes a memory controller hub 420 and an input/output (I/O)) controller hub 422. A memory 406 and a graphics adapter 412 are coupled to the memory controller hub 420, and a display 418 is coupled to the graphics adapter 412. A storage device 408, an input device 414, and network adapter 416 are coupled to the I/O controller hub 422. Other embodiments of the computer 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of pointing device, a keyboard, or some combination thereof, and is used to input data into the computer 400. In some embodiments, the computer 400 may be configured to receive input (e.g., commands) from the input interface 414 via gestures from the user. The network adapter 416 couples the computer 400 to one or more computer networks.

The graphics adapter 412 displays representation, graphs, tables, and other information on the display 418. In various embodiments, the display 418 is configured such that the user (e.g., data scientists, data owners, data partners) may input user selections on the display 418 to, for example, predict immune responses across a particular anatomical compartment or order any additional exams or procedures. In one embodiment, the display 418 may include a touch interface. In various embodiments, the display 418 can show one or more predicted immune responses of an immune system against a HBV. Thus, a user who accesses the display 418 can inform the subject of the predicted immune responses against a HBV.

The computer 400 is adapted to execute computer program modules for providing functionality described herein. In various embodiments, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computers 400 used by the entities of FIG. 1A or 1B can vary depending upon the embodiment and the processing power required by the entity. For example, the hepatitis B virus response system 130 can run in a single computer 400 or multiple computers 400 communicating with each other through a network such as in a server farm. The computers 400 can lack some of the components described above, such as graphics adapters 412, and displays 418.

### V. Systems

Further disclosed herein are systems for implementing FL models for predicting performance of clinical trial sites. In various embodiments, such a system can include at least the hepatitis B virus response system 130 described above in FIG. 1A. In various embodiments, the hepatitis B virus response system 130 is embodied as a computer system, such as a computer system with example computer 400 described in FIG. 4.

In various embodiments, the term “module” is used in the context of systems disclosed herein and refers to an analytical component. In various embodiments, the term “engine” is used in the context of system disclosed herein and refers to an analytical component. An example analytical component may be hardware, such as computational hardware or network hardware. In various embodiments, analytical components may be co-located with one another (e.g., geographically located in proximity to one another). In some embodiments, analytical components may be remotely located from each other (e.g., provided as remote services or executed on remotely located computers connected by a network).

In various embodiments, modules and/or engines can be separate analytical components of one or more systems. In various embodiments, modules and/or engines can be analytical components that operably function together. For example, an engine may be an analytical component of a module. For example, as described herein, a model deployment module can comprise one or more prediction engines. In another example, a response characterization module can comprise one or more response characterization engines. In another example, a model building module can comprise: an input engine, a prediction engine, an evaluation engine, and/or a characterization engine.

### Examples

### I. Example QSP Model

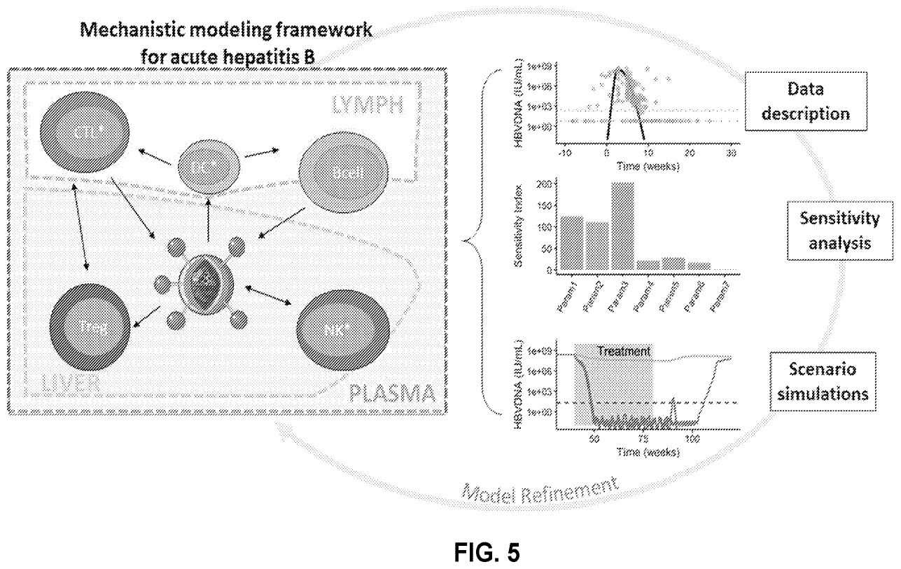

FIG. 5 illustrates example development of a QSP model. FIG. 6 illustrates a schematic representation of the QSP model. The QSP model was developed to consider interaction across 3 compartments: liver (yellow area in FIG. 6), lymph (green area in FIG. 6) and plasma (orange area in FIG. 6) as described herein. The solid lines indicate processes of synthesis, degradation or transport that impact on entities levels. The dotted lines indicate stimulatory (grey area in FIG. 6) or inhibitory (red area in FIG. 6) effects.

### II. Developing and Evaluating a QSP Model

### I.A. Description of the Mathematical Model

Focusing model scope on acute HBV response characterization, the QSP model structure proposed here was based on a topological representation as described in Asin-Prieto E, Parra-Guillen Z P, Mantilla J D G, Vandenbossche J, Stuyckens K, et al (2019) Immune network for viral hepatitis B: Topological representation. Eur J Pharm Sci 136:104939. The topological network (e.g., as shown in FIG. 6) includes the interaction between the virus and key players of the innate, adaptive, and immunoregulatory system across 3 relevant compartments: liver (LV), plasma (PL) and lymph node (LN), as described below.

### I.A.1 Viral Dynamics

The QSP model assumes that a system was initiated with a viral load arriving to the liver. After viral inoculation, HBV can infect healthy hepatocytes (Hep) and be cleared or be distributed through plasma. In turn, infected hepatocytes (iHep) can produce more virus, but also HBsAg that can also be distributed to plasma. All hepatocytes (healthy and infected) were subject to natural death, thus producing debris hepatocytes (dHep), responsible for the production of the hepatic damage biomarker alanine aminotransferase (ALT). Given that HBV is not considered a cytopathic virus and that the number of total hepatocytes (Hepₜₒₜ) is not expected to significantly vary during the acute setting, a quick equilibrium between hepatocyte death and generation was assumed, thus Hep was the difference between total and infected hepatocytes.

### I.A.2. Innate Response

To characterize the innate response, a pool of naïve dendritic cells (DCs) and natural killer (NK) cells with a zero-order synthesis rate in plasma, which accounted for bone marrow formation and distribution from plasma to liver, was assumed. Upon virus recognition, liver DCs were then activated (DC*). A fraction of these DCs*, representing plasmacytoid DCs (pDCs), produce interferon α (IFNα), a cytokine known to inhibit viral replication as well as promote NK cell activation (NK*). Similar to DC*, NK* cells produced IFNγ, which can also inhibit viral replication. In addition, a fraction of these NK*, accounting for NK cells expressing TRAIL, was also able to induce direct iHep death.

On the other hand, a decrease in IFNα synthesis, triggered by HBsAg, was implemented in the model to acknowledge the capability of the virus to limit the innate response against HBV.

### I.A.3. Adaptive Response

DCs* act as the link between innate and adaptive response by triggering cellular and humoral events. In the case of the cellular response, DCs* could migrate directly from liver to lymph tissues, where they triggered the activation and recruitment process of naïve CD8+ T cells to become CD8+ antigen-specific cytotoxic T lymphocytes (CTL). Upon antigen presentation by DCs* to CTL, HBV-specific CTL (CTL*) are generated. These CTL* distribute from lymph node, through plasma to the liver where they exerted a non-cytotoxic antiviral activity via the production of IFNγ, as well as a direct cytotoxic activity killing iHep. Both lymphatic and liver CTL* were considered to be capable of self-proliferation up to a maximum level as long as there is viral presentation or viral load. A fraction of the lymph-generated CTL* could evolve to memory CTL (CTLm).

In addition to the cellular adaptive response, the model also accounted for the HBV-specific humoral response. The presence of DCs* in the lymph node triggered the activation of an existing pool of naïve B cells, which would then convert to plasmablasts (PBs) and initiate a maturation process, until plasma cells (PCs) were obtained. Two populations of PCs were considered in the model—short-lived PCs (SPCs) and long-lived PCs (LPCs)—to enable for a sustained antibody response. Both PCs could then distribute to plasma where they produced specific surface antibodies (anti-HBs) that increased the clearance of HBsAg and viral particles (HBV), as well as antibodies against core antigen (anti-HBc).

The time course of CD4+ lymphocytes was not explicitly included in the model despite their regulator role of effector response, as they were not considered a limiting factor of the immune response in this specific disease.

### I.A.4. Immunotolerant Response

Given the immune tolerogenic nature of the liver, DCs* can activate the generation of liver regulatory T cells (Treg), differentiating from a liver pool of Th0 in order to control the immune response. Treg can self-proliferate as long as a cellular response (CTL*) is still present, and limit liver cellular response upon the induction of CTL exhaustion.

Myeloid-derived suppressor cells (MDSCs) were not included at this stage given the scope of the model and the limited information available from a modeling perspective during the acute phase of hepatitis B. Therefore, assuming that Treg represent both immunotolerant effects.

### I.B. Model Building

Ordinary differential equations (ODEs) were used to describe the time course of the system components across the 3 identified compartments: LV, PL, and LN. In general, synthesis characterized by k_{syn} rate constants was implemented for all components except for infected hepatocytes, for which infection was considered and modeled through a k_{inf} rate constant. Similarly, degradation or death, controlled by k_{deg} and k_{death} rate constants, were specified for all molecular and cellular components, respectively. In addition, activation, exhaustion, or lytic processes represented by k_{act}, kₑₓₕ, and k_{lytic} rate constants were considered. Finally, distribution between compartments was also accounted for when biologically needed (e.g., DC* distribute to lymphoid tissue to active CD8+ cells, and these activated CD8+ reached the liver through the blood stream).

The different biological processes previously detailed (e.g., synthesis, degradation, distribution) were implemented using zero-, first- or second-order rate constants. Michaelis-Menten or Hill kinetic functions were also implemented to account for saturation processes.

The equation below, which describes the temporal course of iHep, is provided as an example.

$$\begin{matrix}{Infection of healthy}_{\text{?}} & {Nat}_{\text{?}}\end{matrix}$$

$$\frac{d iHep}{dt} = {k}_{inf} \times {HBV}_{LV} \times Hep - {k}_{{death}_{Hep}} \times iHep - {k}_{{lytic}_{CTL}} \times {CTL}^{*} \times iHep - {k}_{lytic \_ NK} \times {NK}^{TRAIL} \times iHep$$

$$\begin{matrix}{CTL - mediated}^{\text{?}} & {NK -}^{\text{?}}\end{matrix}$$

The complete set of equations can be found in the Supplementary Appendix 1. The final model accounted for a total of 32 biological entities across ≥1 compartment, described through 41 ODEs and 6 analytical equations. Note that due to data limitations, proportionality was assumed across some entities if specific rates were not required (e.g., IFNs in plasma—potentially useful biomarkers—were assumed to reflect liver quantities after volume correction). Viral replication was considered negligible when <1 hepatocyte was infected.

Mathematical equations were implemented and the dynamics of the different components were simulated using the Simbiology® toolbox from Matlab® (R2019a).

### I.C. Model Parametrization and Initial Conditions

Several methods were applied to provide adequate initial conditions for model entities and parameter estimates for the reactions (n=103). We can differentiate between 2 types of parameters: those that reflect physiological conditions (e.g., organ volumes or entity levels at baseline) and those parameters describing the rate of the different biological and disease processes. Below, the different methods were described with the associated assumptions and the degree of uncertainty. A special effort was made to avoid data not coming from human origin and to select mechanistic and robust data from the literature.
1) Physiological values extracted directly from the literature: This methodology was primarily used for estimates or parameters that were well established and assumed to be physiologically plausible. As an example, the liver volume was set to 1500 mL, rounding the value provided by Irving et al (28) (1470 cm³).
2) Physiological values calculated from published information: Data were obtained from 1 or more sources and used for the derivation of the parameters. One example is the derivation of the volume of plasma in the body, assuming that around 60% of the total blood volume (ca. 5 L) corresponds to plasma, leading to a derived plasma volume of 3000 mL (29). In another example, the number of Hepₜₒₜ was derived assuming that the hepatic volume is 1470 cm³ (28), the volume of a single hepatocyte is 4900 μm³ (30), and the parenchymal cell percentage of the total liver is 80%. The total hepatocyte cell number was estimated to be 2.4×10¹¹ cells.

$${Hep}_{tot} = \frac{1470 {cm}^{3} \times 0.8 \times {10}^{12} \frac{{μm}^{3}}{{cm}^{3}}}{4900 \frac{{μm}^{3}}{cell}} = 2.4 \times {10}^{11} cells$$

3) Derived parameters or initial conditions from the implemented QSP model: Some of the parameters were directly derived from model equations to ensure homeostasis in the absence of viral infection. This was the case for the daily production of naïve DCs or NK cells in plasma.
4) Reused model parameter estimates from previously published models: In these cases, the estimates were obtained from previously published theoretical or applied models. Special attention was paid to evaluate the model assumptions and the nature of the data or references used for model development. When available, more than 1 reference was consulted to increase confidence in the parameter value. As an example, the infection rate constant was obtained from a previous model fitted to clinical data from hepatitis B infected patients under treatment (19). This model included uninfected and infected hepatocytes and the virus. The infection rate constant was estimated 3×10¹⁰ mL/(virion*day) (ranging from 0.7×10¹⁰ to 6.7×10¹⁰). Similarly, other authors used a value for the infection rate constant within the same range (4×10¹⁰ mL/[virion*day]) when fitting data from patients under treatment and modelling uninfected and infected hepatocytes, viruses, and effector cells (31).
5) Parameter estimation from literature experimental data: When parameterization of the biological processes was not directly available, but experimental data quantitatively characterizing the individual process (commonly through in vitro designs) was identified, data from the original publication was extracted or digitalized using WebPlotDigitizer 3.8 and fitted to a model, as previously shown (32). For example, this approach was used to identify the IFNγ liver concentration inhibiting 50% of HBV synthesis (IFNγ_{50_HBV}) based on experimental data from 2 publications (33,34), where the inhibition of HBV replication in a liver cell line was explored in vitro at different IFNγ levels and under different conditions. The inhibitory model developed was further challenged using additional validation data corroborating the noncytolytic effect of IFNγ on viral replication (35).
6) Calibrated parameters: Unfortunately, quantitative information to characterize all described biological interactions was not available. In those situations, arbitrary values (n=6) or fine-tune estimates (n=4) within plausible ranges were used to achieve a desired behaviour. For example, Hill functions were implemented on some processes to act like enablers, activating or deactivating certain processes in the presence or absence of a minimum level of a second component (e.g., activation of DCs in the presence of viral levels). The implications of these estimates in model performance were later explored through sensitivity analyses (see below).

In Table S1 (see Appendix section), all 103 parameters used in the model were listed along with their value and range if available, the methodology used for their extraction, and the references.

### I.D. Model Evaluation

Model performance was evaluated at 2 levels. First, the capability of the model to reproduce the temporal and sequential appearance of the different entities in a biological and plausible manner was evaluated and compared to general disease progression knowledge. Then, typical (median) model predictions were compared to clinical data extracted from 4 publications where the time course of different biological markers in acute patients was reported. These biological markers included HBV DNA circulating levels, as well as ALT, HBsAg activated CTL, or IFNα levels in plasma. Data were digitalized from original figures using WebPlotDigitizer 3.8. An overview of the clinical studies can be found in Table 1 below. To compare model predictions to observed data, time profiles were normalized with respect to HBV DNA peak, as infection time is unknown in most real cases.

TABLE 1
Overview of clinical data studies used for model evaluation.

| Reference | Brief description | Measured variables |
|---|---|---|
| Webster 2000 (58) | 5 patients identified during | HBV DNA (pg/mL) (n = 5) |
| | incubation period | ALT (U/L) (n = 5) |
| | Day 0 based on the most recent | HBsAg (boolean) (n = 5) |
| | possible time point of infection | IgM anti-HBc (Boolean) (n = 5) |
| | | NK cells (cells/mL) (n = 3) |
| | | HBV-specific CD8+ (cells/mL) (n = 3) |
| Dunn 2009 (47) | 21 patients with acute HBV | HBV DNA (IU/mL) (n = 9) |
| | sampled during pre- | ALT (IU/L) (n = 8) |
| | symptomatic phase | HBsAg (boolean) (n = 6) |
| | Day 0 on first symptomatic day | IgM anti-HBc (Boolean) (n = 5) |
| | | IFNa (pg/mL) (n = 3) |
| | | HBV-specific CD8+ (cells/mL) (n = 4) |
| Fisicaro 2009 (46) | 2 blood donors found to have | HBV DNA (IU/mL) (n = 2) |
| | seroconverted during virological | ALT (IU/L) (n = 2) |
| | screening every 3 months | HBsAg (boolean) (n = 2) |
| | Day 0 assumed at previous | anti-HBs (U/L) (n = 2) |
| | serological screening day | anti-HBc (Boolean) (n = 2) |
| Chulanov 2003 (37) | 21 patients hospitalized with | HBV DNA (ge/mL) (n = 21) |
| | suspected acute viral hepatitis | ALT (ULN) (n = 21) |
| | and confirmed of HBV | HBsAg (μg/mL) (n = 21) |
| | monoinfection | |
| | Day 0 based on first | |
| | symptomatic (illness) day | |

### I.E. Parameter Analyses

Robustness of the final model and impact of the different implemented processes on model entities was assessed through a local sensitivity analysis using the complex-step approximation method (MATLAB, R2019a). The fully normalized sensitivity profiles over time for all the model components were computed and the integral was reported.

In addition, a parameter scan analysis was performed to assess the impact of individual parameter variations (+/−50%) on relevant end points and identify those processes that could drive the system towards a chronic infection situation. Infection resolution was considered if plasma HBV DNA levels fell below 20 IU/mL (i.e. undetectable levels) and change in time to resolution was computed taking into consideration the maximum simulation time of 45 weeks.

### I.F. Exploring Model Behaviour

### I.F.1 Relevance of Immune Pathways

A “knock-out” analysis was performed to evaluate the relative importance of the innate (k_{act_NK}=0 or k_{act_DC}=0), cellular (k_{act_CTL}=0) or humoral (k_{act_Bcell}=0) immune response components on the profile of HBVDNA, the main marker of adequate clearance of the infection. The role of the immunoregulatory response was also explored by modifying the sensitivity of regulatory cells proliferation to CTL presence (CTL_{50_Treg_prol}) or the inhibitory effect of regulatory cells on CTL response (Treg_{50_Treg_exh}).

### I.F.2 Chronicity

The capability of the model to mimic acute status or development of chronicity was evaluated at a population level computing the percentage of subjects self-resolving the disease (i.e., HBV DNA<20 IU/mL at week 45). To do so, a virtual population (n=500) was generated assuming a log normal distribution with 30% variability of the most influential parameters identified during a parameter scan (sensitivity index above 50 units). The distributions were truncated to limit the simulated values to the reported ranges for the different model parameters (Table S1). The process was repeated 100 times to obtain a confidence interval around the percentage of self-resolving patients.

### III. Example Results from a QSP Model

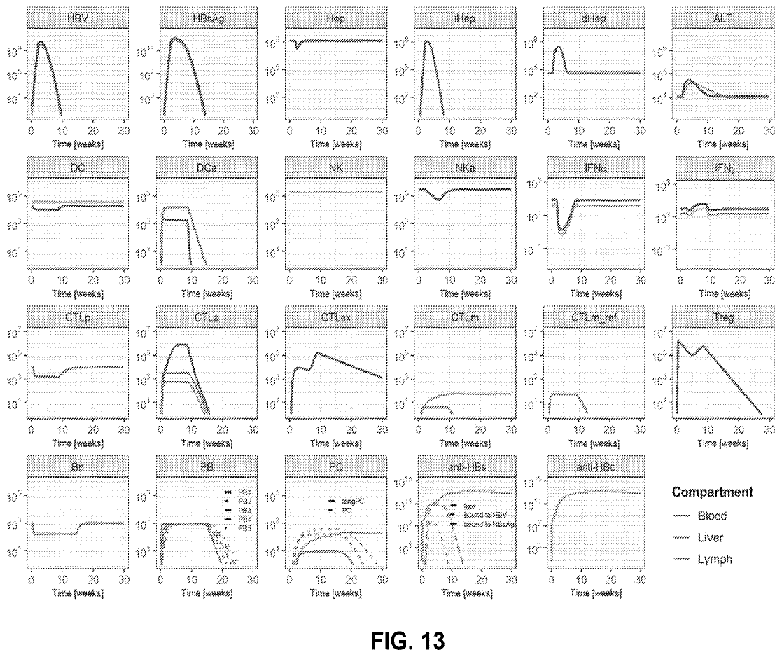

The final HBV model comprised a total of 41 ODEs and 6 analytical equations defined by 84 parameters to enable the prediction of the time course of main viral and liver components, as well as cellular and molecular entities of the innate and adaptive response across 3 compartments: liver, plasma and lymph tissue. Predicted time profiles for the different model entities across the 3 compartments (blood, liver and lymph)) are shown in FIG. 13.

### III.A. Model Evaluation

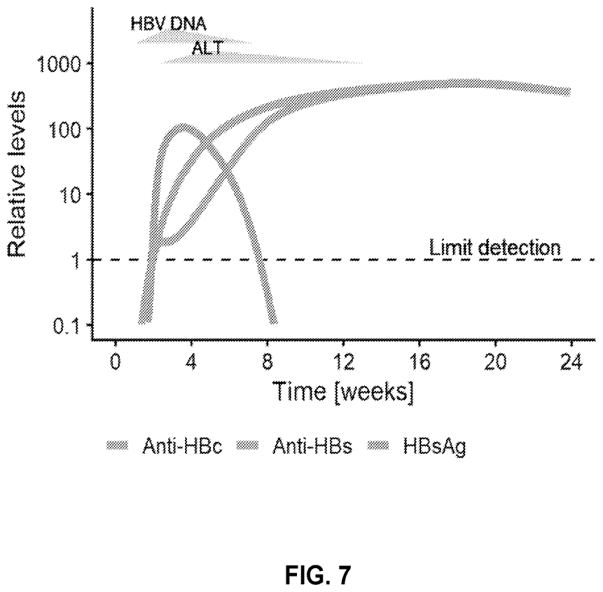

The model was capable of reproducing the general knowledge regarding the typical time course of the acute disease in patients as shown in FIG. 7. FIG. 7 illustrates disease course of AHB, in which the QSP model predicted time course of common biomarkers of AHB disease normalized to their limit of detection. HBV DNA and ALT triangles in FIG. 7 represent the span and the time of peak levels, as describe herein.

Although a quick disease onset of 3 weeks was predicted after viral infection, the model predicted that viral levels <200 IU/mL would be reached 52 days after viral peak, and complete viral eradication (<20 IU/mL) in approximately 8 weeks. Similarly, peak in ALT levels is predicted 2 weeks after HBV DNA peak, and 3 weeks after the appearance of detectable HBsAg (>0.1 ng/mL). Levels of HBsAg remain above that the cut-off for up to 10 weeks after inoculation. Antibody response was predicted to be delayed on infection, with anti-HBs levels arising above the protection threshold (10 IU/mL) between weeks 7-8 after infection, once HBsAg levels are undetectable.

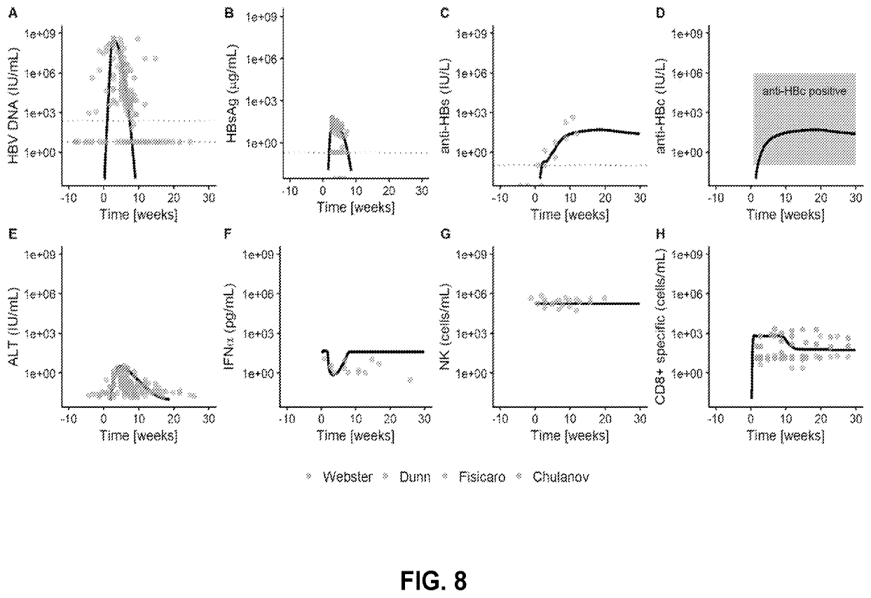

FIG. 8 illustrates evaluation of the final QSP model associated with AHB. The model predictions (solid line) versus data (points) extracted from different clinical studies. Further details on data availability and study characteristics are shown in Table 1 and described herein. As shown in FIG. 8, the proposed model was able to capture the time course of relevant clinical biomarkers from patients with acute HBV infection extracted from several publications, as shown in FIG. 8. To note, only ALT levels extracted from clinical data were used to calibrate ALT-related model parameters using the final model structure; the rest of the clinical data were used as a validation set.

### III.B. Parameter Analyses

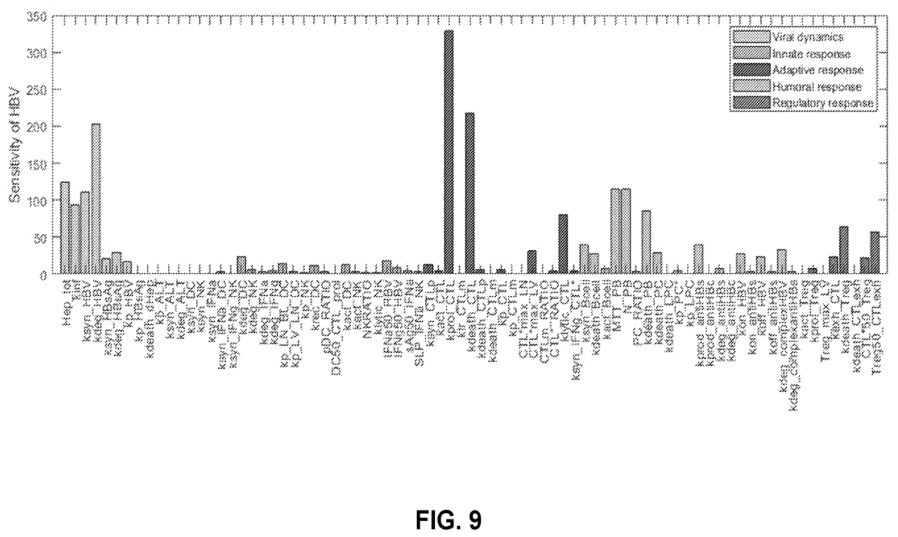

FIG. 9 illustrates results from a local sensitivity analysis. As shown in FIG. 9, sensitivity index for the different model parameters grouped by immune pathway computed as the integral of the fully normalized sensitivity profiles over HBV DNA time profiles. The model parameters were described herein (e.g., parameters 120 in FIG. 1A) and shown in Supplementary Table 1.

Parameters governing the proliferation/death of CTL, followed by those directly affecting viral dynamics (infectivity, target cells, and viral synthesis) and the parameters regulating the appearance of PCs (mean transit time and number of transits of PBs) were the most influential on HBV levels.

To evaluate the impact of the above processes not only on the time course of hepatitis B infection, but also the probability of becoming chronic (i.e., time to cure), a parameter scan was performed.

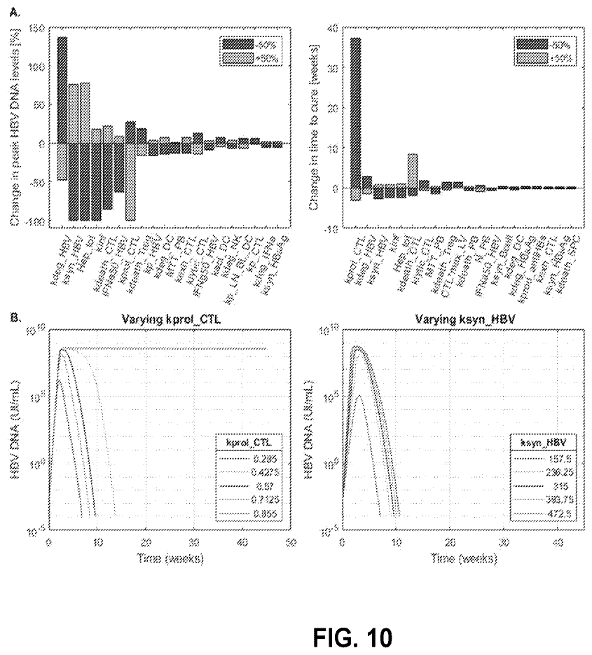

FIG. 10A illustrates impact of 50% change on peak HBV DNA levels or time to cure (defined as HBV DNA<20 IU/L & maximum simulation time of 45 weeks) when varying one parameter at a time. FIG. 10B illustrates impact of varying CTL proliferation rate constant (kprol_CTL) or HBV synthesis constant (ksyn_HBV) on the time course of circulating DNA viral levels (HBV DNA). Further details about the parameters were described herein and in Supplementary Table 1.

The 20 most influential parameters on peak viral levels and time to cure are shown in FIG. 10A. The processes influencing peak or area under the curve levels (not shown) are in close agreement to those identified in sensitivity analysis. However, under the acute scenario, only the change in the proliferation capability of T cells was able to switch from responder to non-responder, while moderate changes in the rest of meaningful parameters such as viral replication capability (k_{syn_HBV}) impact the maximum levels or time to cure, but not the ultimate response (FIG. 10B).

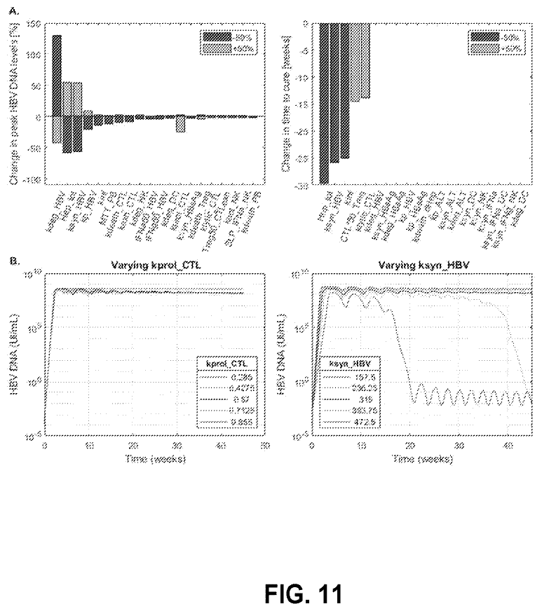

FIGS. 11A-B illustrates parameter scan analysis under a theoretical chronic scenario (increased Treg sensitivity). FIG. 11A illustrates impact of 50% change on peak HBV DNA levels or time to cure (defined as HBV DNA<20 IU/L & maximum simulation time of 45 weeks) when varying one parameter at a time. FIG. 11B illustrates impact of varying CTL proliferation rate constant (k_{prol_CTL}) or HBV synthesis constant (k_{syn_HBV}) on the time course of circulating DNA viral levels (HBV DNA).

### III.C. Exploring Model Behaviour

### III.C.1 Contribution of Immune Pathways

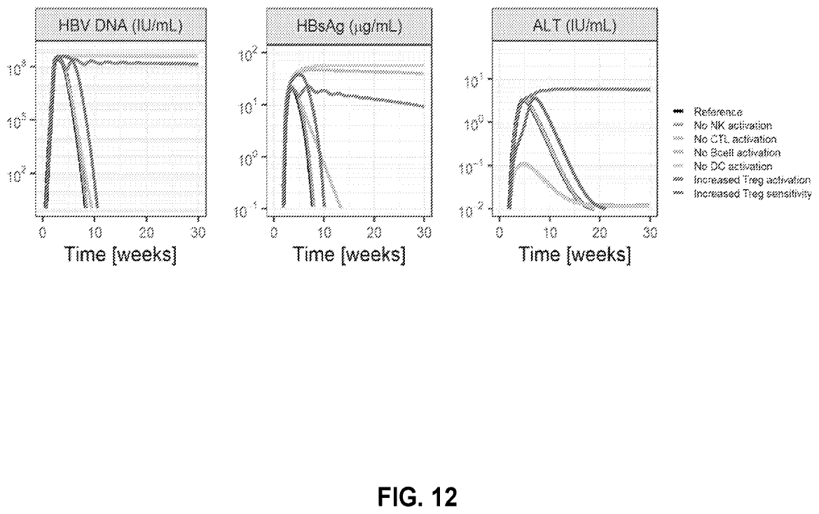

The relative contribution of each of the implemented pathways on the time profile of relevant disease biomarkers—viral load, HBVs antigens, and ALT—was explored by individually suppressing or activating their triggers one at a time (FIG. 12). FIG. 12 illustrates a knock-out analysis based on model predicted time course of viral load (HBV DNA), surface hepatitis B antigen (HBsAg), and alanine aminotransferase (ALT) in blood under different knock-out scenarios: no perturbation (reference), no NK activation (k_{act_NK}=0), no CTL activation (k_{act_CTL}=0), no Bcell activation (k_{act_Bcell}=0), no DC activation (k_{act_DC}=0), increased Treg activation (CTL_{50_Treg_prol}=200000) or increased Treg sensitivity to CTL exhaustion (Treg_{50_CTL_exh}=200000).

Consistent with the parameter analyses results, blocking the activation of NK cells had no impact on viral dynamics or course. Similarly, blocking B cell activation or increasing the inhibitory effects of regulatory T cells by a factor of 5 had some impact on slowing down the elimination of antigen or delaying the start of viral clearance respectively, but did not change the ultimate outcome, AHB disease resolution.

On the other hand, when blocking the activation of effector cells (CTL) or the antigen presentation to DC to initiate the response, an outcome of CHB scenario was predicted. A similar outcome was observed when the sensitivity to the activation of the immunoregulatory response was increased by a factor of 5, although with a sustained hepatic damage trigger by the remaining CTLs.

### III.C.2. Chronicity

To further explore the captured behaviour described under model evaluation, the capability of the model to predict self-resolution or evolution to a chronic status was evaluated at a population level. Those parameters exhibiting higher impact on the sensitivity analysis—11 in total—were selected and varied to simulate a virtual population: viral infectivity (k_{inf}), replication (k_{syn_HBV}) and degradation (k_{deg_HBV}) rate constants representing viral dynamics; CTL proliferation (k_{prol_CTL}), degradation (k_{deg_CTL}) and lytic activity (k_{lytic_CTL}) rate constants representing the cellular adaptive response; PB death rate constant (k_{death_PB}), PB mean transit time (MTT_{PB}), and number of transit compartments (NN_{PB}) regarding the humoral response; and Treg death rate (k_{death_Treg}) and Treg levels triggering 50% of maximum rate of CTL exhaustion (Treg_{50_CTLexh}) representing the immunoregulatory response. The maximum (total) number of hepatocytes was not varied, as it rather represents a physiological parameter, which can be considered constant. In addition, no parameters regarding the innate response were selected due to their limited impact.

The virtual simulated population provided a simulated percentage of chronicity of 4.6% with a 95% confidence interval of 3.0-6.4%.

### IV. Summary and Discussion of Examples

Different mathematical models have been developed previously for acute HBV; however, they have focused on certain aspects of the immune response in isolation. The quantitative QSP model as described herein aimed to integrate in a single framework the role of different relevant immune response components—innate, adaptive and immunoregulatory—involved in HBV viral clearance, across multiple compartments (plasma, liver, and lymph nodes).

To build the final QSP model, information from multiple sources including existing models, clinical quantitative knowledge, and in vitro experiments were integrated. Despite limitations, when simulating a virtual population that takes into account reasonable variability (30%) on influential model parameters and using the modeling framework developed for the acute scenario, development of chronic HBV infection was predicted for 4.6% of the simulated population.

$$SUPPLEMENTARY APPENDIX$$

$$I . Model equations$$

$$Liver$$

$$Hep = {Hep}_{tot} - iHep \qquad ( equation 1 )$$

$$( equation 2 )$$

$$\frac{d iHep}{dt} = {k}_{inf} \times {HBV}_{LV} \times Hep - {k}_{death \_ Hep} \times iHep - {k}_{lytic \_ CTL} \times {CTL}_{LV}^{*} \times iHep - {k}_{lytic \_ NK} \times {NK}^{TRAIL} \times iHep$$

$$( equation 3 )$$

$$\frac{d dHep}{dt} = {k}_{death \_ Hep} \times ( Hep + iHep ) + {k}_{lytic \_ CTL} \times {CTL}_{LV}^{*} \times iHep + {k}_{lytic \_ NK} \times {NK}^{TRAIL} \times iHep - {k}_{death \_ dHep} \times dHep$$

$$( equation 4 )$$

$$\frac{d {HBV}_{LV}}{dt} = {k}_{syn \_ HBV} \times iHep \times ( 1 - \frac{IFN α}{IFN α + IFN {α}_{50 \_ HBV}} ) \times ( 1 - \frac{IFN γ}{IFN γ + IFN {γ}_{50 \_ HBV}} ) - {k}_{inf} \times {HBV}_{LV} \times Hep - {k}_{LV \_ PL \_ HBV} \times {HBV}_{LV} + {k}_{PL \_ LV \_ HBV} + {HBV}_{PL} \times \frac{{V}_{PL}}{{V}_{LV}} - {k}_{deg \_ HBV} \times {HBV}_{LV}$$

$$( equation 5 )$$

$$\frac{d {HBsAg}_{LV}}{dt} = {k}_{syn \_ HBsAg} \times iHep \times ( 1 - \frac{IFN α}{IFN α + IFN {α}_{50 \_ HBV}} ) \times ( 1 - \frac{IFN γ}{IFN γ + IFN {γ}_{50 \_ HBV}} ) - {k}_{LV \_ PL \_ HBsAg} \times {HBsAg}_{LV} + {k}_{PL \_ LV \_ HBsAg} \times {HBsAg}_{PL} \times \frac{{V}_{PL}}{{V}_{LV}} - {k}_{deg \_ HBsAg} \times {HBsAg}_{LV}$$

$$( equation 6 )$$

$$\frac{d {DC}_{LV}}{dt} = {k}_{PL \_ LV \_ DC} \times {DC}_{PL} \times \frac{{V}_{PL}}{{V}_{LV}} - {k}_{act \_ DC} \times \frac{{HBV}_{LV}^{4}}{{HBV}_{LV}^{4} + {HBV}_{50 \_ DC}^{4}} \times {DC}_{LV} - {k}_{death \_ DC} \times {DC}_{LV}$$

$$( equation 7 )$$

$$\frac{d {DC}_{LV}^{*}}{dt} = {k}_{act \_ DC} \times \frac{{HBV}^{4}}{{HBV}^{4} + {HBV}_{50 \_ DC}^{4}} \times {DC}_{LV} - {k}_{death \_ DC} \times {DC}_{LV}^{*} - {k}_{LV \_ LN {\_ DC}^{*}} \times {DC}_{LV}^{*}$$

$$( equation 8 )$$

$$\frac{d IFN {α}_{LV}}{dt} = {k}_{syn \_ IFN α} + {k}_{syn \_ IFN {α\_ DC}^{*}} \times {DC}_{LV}^{*} \times {pDC}_{RATIO} \times ( 1 - \frac{{HBsAg}_{LV}}{{HBsAg}_{LV} + {HBsAg}_{{LV}_{50 \_ IFN α}}} ) - {k}_{deg \_ IFN α} \times IFN {α}_{LV}$$

$$( equation 9 )$$

$$\frac{d {NK}_{LV}}{dt} = {k}_{PL \_ LV \_ NK} \times {NK}_{PL} \times \frac{{V}_{PL}}{{V}_{LV}} - {k}_{act \_ NK} \times {NK}_{LV} \times ( 1 + {SLP}_{IFN α\_ NK} \times IFN {α}_{LV} ) - {k}_{death \_ NK} \times {NK}_{LV}$$

$$( equation 10 )$$

$$\frac{d {NK}_{LV}^{*}}{dt} = {k}_{act \_ NK} \times {NK}_{LV} \times ( 1 + {SLP}_{IFN α\_ NK} \times IFN {α}_{LV} ) - {k}_{death \_ NK} \times {NK}_{LV}^{*}$$

$${NK}^{TRAIL} = {NK}_{RATIO} \times {NK}_{LV}^{*} \qquad ( equation 11 )$$

$$( equation 12 )$$

$$\frac{d {IFN}_{γ LV}}{dt} = {k}_{syn \_ IFN γ} + {k}_{syn \_ IFN γ \_ NK} \times {NK}_{LV}^{*} + {k}_{syn \_ IFN γ \_ CTL} \times {CTL}_{LV}^{IFN γ +} - {k}_{deg \_ IFN γ} \times IFN {γ}_{LV}$$

$${CTL}_{LV}^{IFN γ +} = {CTL}_{LV}^{*} \times {CTL}_{RATIO}^{*} \qquad ( equation 13 )$$

$$( equation 14 )$$

$$\frac{d {CTL}_{LV}^{*}}{dt} = {k}_{PL \_ LV {\_ CTL}^{*}} \times {CTL}_{PL}^{*} \times \frac{{V}_{PL}}{{V}_{LV}} + {k}_{prol \_ CTL} \times {CTL}_{LV}^{*} \times ( 1 - \frac{{CTL}_{LV}^{*} + {CTL}_{exh}}{{CTL}_{max \_ LV}^{*}} ) \times ( \frac{{HBV}_{LV}^{4}}{{HBV}_{LV}^{4} + {HBV}_{50 \_ CTL \_ prol}^{4}} ) - {k}_{{exh \_ CTL}^{*}} \times {CTL}_{LV}^{*} \times ( \frac{{Treg}_{LV}^{4}}{{Treg}_{LV}^{4} + {Treg}_{50 \_ CTL \_ exh}^{4}} ) - {k}_{death \_ CTL} \times {CTL}_{LV}^{*}$$

$$( equation 15 )$$

$$\frac{d {CTL}_{exh}}{dt} = {k}_{{exh \_ CTL}^{*}} \times {CTL}_{LV}^{*} \times ( \frac{{Treg}_{LV}^{4}}{{Treg}_{LV}^{4} + {Treg}_{50 \_ CTL \_ exh}^{4}} ) - {k}_{death \_ CTLexh} \times {CTL}_{exh}$$

$$\frac{dTh 0}{dt} = - {k}_{acct \_ Treg} \times Th 0 \times {DC}_{LV}^{*} \qquad ( equation 16 )$$

$$( equation 17 )$$

$$\frac{dTreg}{dt} = {k}_{act \_ Treg} \times Th 0 \times {DC}_{LV}^{*} + {k}_{prol \_ Treg} \times Treg \times ( 1 - \frac{Treg}{{Treg}_{max \_ LV}} ) \times ( \frac{{CTL}_{LV}^{* 4}}{{CTL}_{LV}^{* 4} + {CTL}_{50 \_ Treg \_ prol}^{4}} ) - {k}_{{death}_{Treg}} \times Treg$$

$$( equation 18 )$$

$$\frac{d {ALT}_{LV}}{dt} = {k}_{syn \_ ALT} \times dHep - {k}_{LV \_ PL \_ ALT} \times {ALT}_{LV} - {k}_{deg \_ ALT} \times {ALT}_{LV}$$

$$Plasma$$

$$\frac{d {ALT}_{PL}}{dt} = {k}_{LV\_PL \_ALT} \times {ALT}_{LV} \times \frac{{V}_{LV}}{{V}_{PL}} - {k}_{deg\_ALT} \times {ALT}_{PL} \qquad ( equation 19 )$$

$$( equation 20 )$$

$$\frac{d {HBV}_{PL}}{dt} = {k}_{LV \_ PL \_ HBV} \times {HBV}_{LV} \times \frac{{V}_{LV}}{{V}_{PL}} - {k}_{PL \_ LV \_ HBV} \times {HBV}_{PL} - {k}_{deg \_ HBV} \times {HBV}_{PL} - {k}_{on \_ antiHBs} \times {HBV}_{PL} \times {antiHBs}_{PL} + {k}_{off \_ antiHBs} \times HBV : {antiHBs}_{PL}$$

$$HBVDNA = {HBV}_{BL} \times \frac{1}{5} \qquad ( equation 21 )$$

$$( equation 22 )$$

$$\frac{d {HBsAg}_{PL}}{dt} = {k}_{LV \_ PL \_ HBsAg} \times {HBsAg}_{LV} \times \frac{{V}_{LV}}{{V}_{PL}} - {k}_{PL \_ LV \_ HBsAg} \times {HBsAg}_{PL} - {k}_{deg\_HBsAg} \times {HBsAg}_{PL} - {k}_{on\_antiHBs} \times {HBsAg}_{PL} \times {antiHBs}_{PL} + {k}_{off\_antiHBs} \times HBsAg : {antiHBs}_{PL}$$

$$\frac{d {DC}_{PL}}{dt} = {k}_{syn \_ DC} - {k}_{PL\_LV \_DC} \times {DC}_{PL} - {k}_{death\_DC} \times {DC}_{PL} \qquad ( equation 23 )$$

$$IFN {α}_{PL} = IFN {α}_{LV} \times \frac{{V}_{LV}}{{V}_{PL}} \qquad ( equation 24 )$$

$$\frac{d {NK}_{PL}}{dt} = {k}_{syn\_NK} - {k}_{PL\_LV \_NK} \times {NK}_{PL} - {k}_{death\_NK} \times {NK}_{LV} \qquad ( equation 25 )$$

$$IFN {γ}_{PL} = IFN {γ}_{LV} \times \frac{{V}_{LV}}{{V}_{PL}} \qquad ( equation 26 )$$

$$( equation 27 )$$

$$\frac{d {CTL}_{PL}^{*}}{dt} = {k}_{LN\_PL {\_CTL}^{*}} {CTL}_{LN}^{*} \times \frac{{V}_{LN}}{{V}_{PL}} - {k}_{PL\_LV {\_CTL}^{*}} \times {CTL}_{PL}^{*} - {k}_{{death \_ CTL}^{*}} \times {CTL}_{PL}^{*}$$

$$( equation 28 )$$

$$\frac{d {CTLm}_{PL}}{dt} = {k}_{LN \_ PL \_ CTLm} \times {CTLm}_{LN} \times \frac{{V}_{LN}}{{V}_{PL}} - {k}_{death \_ CTLm} \times {CTLm}_{PL}$$

$$\frac{d {SPC}_{PL}}{dt} = {k}_{LN\_PL \_PC} \times {SPC}_{LN} \times \frac{{V}_{LN}}{{V}_{PL}} - {k}_{death \_ PC} \times {SPC}_{PL} \qquad ( equation 29 )$$

$$\frac{d {LPC}_{PL}}{dt} = {k}_{LN\_PL \_LPC} \times {LPC}_{LN} \times \frac{{V}_{LN}}{{V}_{PL}} - {k}_{death \_ LPC} \times {LPC}_{PL} \qquad ( equation 30 )$$

$$( equation 31 )$$

$$\frac{d {antiHBs}_{PL}}{dt} = {k}_{prod \_ antiHBs} \times ( {PC}_{PL} + {LPC}_{PL} ) - {k}_{deg \_ antiHBs} \times {antiHBs}_{PL} - {k}_{on \_ HBsAg} \times ( {HBV}_{PL} + {HBsAg}_{PL} ) \times {antiHBs}_{PL} + {k}_{off \_ HBsAg} \times ( HBV : {antiHBs}_{PL} + HBsAg : {antiHBs}_{PL} )$$

$$( equation 32 )$$

$$\frac{d {antiHBc}_{PL}}{dt} = {k}_{prod \_ antiHBc} \times ( {PC}_{PL} + {LPC}_{PL} ) - {k}_{deg \_ antiHBc} \times {antiHBc}_{PL}$$

$$( equation 33 )$$

$$\frac{d HBV : {antiHBs}_{PL}}{dt} = {k}_{on \_ antiHBs} \times {HBV}_{PL} \times {antiHBs}_{PL} - {k}_{off \_ antiHBs} \times HBV : {antiHBs}_{PL} - {k}_{deg \_ complexHBV} \times HBV : {antiHBs}_{PL}$$

$$( equation 34 )$$

$$\frac{d HBsAg : {antiHBs}_{PL}}{dt} = {k}_{on \_ antiHBs} \times {HBsAg}_{PL} \times {antiHBs}_{PL} - {k}_{off \_ antiHBs} \times HBsAg : {antiHBs}_{PL} - {k}_{deg \_ complexHBsAg} \times HBsAg : {antiHBs}_{PL}$$

$$Lymph$$

$$\frac{d {DC}_{LN}^{*}}{dt} = {k}_{LV \_ LN {\_ DC}^{*}} \times {DC}_{LV}^{*} \times \frac{{V}_{LV}}{{V}_{LN}} - {k}_{death \_ DC} \times {DC}_{LN}^{*} \qquad ( equation 35 )$$

$$( equation 36 )$$

$$\frac{d {CTL}_{LN}}{dt} = {k}_{syn \_ CTL} + {k}_{rec \_ DC} \times {DC}_{LN}^{*} - {k}_{death \_ CTL} \times {CTL}_{LN} - {k}_{act \_ CTL} \times {CTL}_{LN} \times {DC}_{LN}^{*}$$

$$( equation 37 )$$

$$\frac{d {CTL}_{LN}^{*}}{dt} = {k}_{act \_ CTL} \times {CTL}_{LN} \times {DC}_{LN}^{*} + {k}_{prol \_ CTL} \times {CTL}_{LN}^{*} \times ( 1 - \frac{{CTL}_{LN}^{*}}{{CTL}_{max \_ LN}^{*}} ) \times ( \frac{{( {DC}_{LN}^{*} )}^{4}}{{( {DC}_{LN}^{*} )}^{4} + {DC}_{50 \_ CTL \_ prol}^{4}} ) - {k}_{LN \_ PL \_ CTL} \times {CTL}_{LV}^{*} - {k}_{{death \_ CTL}^{*}} \times {CTL}_{LN}^{*}$$

$$( equation 38 )$$

$$\frac{d {CTLm}_{LN}^{ref}}{dt} = {k}_{{death \_ CTL}^{*}} \times {CTL}_{LN}^{*} \times {CTLm}_{RATIO} - {k}_{tr \_ CTLm} \times {CTLm}_{LN}^{ref} - {k}_{{death \_ CTLm}^{ref}} \times {CTLm}_{LN}^{ref}$$

$$( equation 39 )$$

$$\frac{d {CTLm}_{LN}}{dt} = {k}_{tr \_ CTLm} \times {CTLm}_{LN}^{ref} - {k}_{LN \_ PL \_ CTLm} \times {CTLm}_{LN} - {k}_{death \_ CTLm} \times {CTLm}_{LN}$$

$$( equation 40 )$$

$$\frac{d Bcell}{dt} = {k}_{syn \_ Bcell} - {k}_{act \_ Bcell} \times Bcell \times ( \frac{{( {DC}_{LN}^{*} )}^{4}}{{( {DC}_{LN}^{*} )}^{4} + {DC}_{50 \_ CTL \_ prol}^{4}} ) - {k}_{death \_ Bcell} \times Bcell$$

$$( equation 41 )$$

$$\frac{d PB 1}{dt} = {k}_{act \_ Bcell} \times Bcell \times ( \frac{{( {DC}_{LN}^{*} )}^{4}}{{( {DC}_{LN}^{*} )}^{4} + {DC}_{50 \_ CTL \_ prol}^{4}} ) - \frac{{N}_{PB}}{{MTT}_{PB}} \times PB 1 - {k}_{death \_ PB} \times PB 1$$

$$\frac{d PB 2}{dt} = \frac{{N}_{PB}}{{MTT}_{PB}} \times ( 2 \times PB 1 - PB 2 ) - {k}_{death \_ PB} \times PB 2 \qquad ( equation 42 )$$

$$\frac{d PB 3}{dt} = \frac{{N}_{PB}}{{MTT}_{PB}} \times ( 2 \times PB 2 - PB 3 ) - {k}_{death \_ PB} \times PB 3 \qquad ( equation 43 )$$

$$\frac{d PB 4}{dt} = \frac{{N}_{PB}}{{MTT}_{PB}} \times ( 2 \times PB 3 - PB 4 ) - {k}_{death \_ PB} \times PB 4 \qquad ( equation 44 )$$

$$\frac{d PB 5}{dt} = \frac{{N}_{PB}}{{MTT}_{PB}} \times ( 2 \times PB 4 - PB 5 ) - {k}_{death \_ PB} \times PB 5 \qquad ( equation 45 )$$

$$( equation 46 )$$

$$\frac{d SPC}{dt} = 2 \times \frac{{N}_{PB}}{{MTT}_{PB}} \times PB 5 \times ( 1 - {PC}_{RATIO} ) - {k}_{LN \_ PL \_ PC} \times SPC - {k}_{death \_ PC} \times SPC$$

$$( equation 47 )$$

$$\frac{d SPC}{dt} = 2 \times \frac{{N}_{PB}}{{MTT}_{PB}} \times PB 5 \times {PC}_{RATIO} - {k}_{LN \_ LPL \_ PC} \times LPC - {k}_{death \_ LPC} \times LPC$$

TABLE S1
II. Definition, estimates, ranges and sources of the different model parameters

| | | | | | |
|---|---|---|---|---|---|
| Parameter | Units | Definition | Value | Range | Ref. |

Volumes

| | | | | | |
|---|---|---|---|---|---|
| V_{LV} | mL | Liver volume | 1500 | — | (1) |
| V_{PL} | mL | Plasma volume | 3000 | — | (2) |
| V_{LN} | mL | Lymph nodes volume | 730 | — | (3) |

Synthesis, proliferation, recruitment & activation rate constants

| | | | | | |
|---|---|---|---|---|---|
| K_{inf} | mL/virion/d | Hepatocytes infection rate constant | 3 × 10⁻¹⁰ | (3-4) × 10⁻¹⁰ | (4, 5) |
| K_{syn}_HBV | virion/cell/d | HBV synthesis rate constant by iHep | 315 | 71-1000 | (4, 6)¹ |
| K_{syn}_HBsAg | molec/cell/d | HBsAg synthesis rate constant by iHep | 45165 | — | Derived² |
| K_{act DC} | 1/d | Naïve DC activation rate constant | 0.2 | 0.2-0.4 | (7) |
| K_{syn}_IFNα | pg/mL/d | IFNα independent production | 459.2 | — | Derived³ |
| K_{syn IFNα} _{DC*} | pg/cell/d | IFNα produced by pDC fraction of DC* | 0.1 | 0.018-2.97 | (8-13) |
| K_{act}_NK | 1/d | Naïve NK activation rate constant | 1.82 × 10⁻⁵ | — | Derived⁴ |
| K_{syn}_IFNγ_NK | pg/cell/d | IFNγ produced by increments on NK* | 0.00334 | — | Derived⁵ |
| K_{syn}_IFNγ_CTL | pg/cell/d | IFNγ produced by liver IFNγ+ CTL | 0.0034 | — | (14)⁶ |
| Kₚᵣₒₗ_CTL | 1/d | Proliferation of active CTL in liver and lymph node | 0.57 | 0.4-0.9 | (15)(7)⁷ |
| K_{act Treg} | mL/cell/d | Activation of Treg in the liver | 0.0256 | — | (16) |
| Kₚᵣₒₗ_Treg | 1/d | Proliferation of Treg in the liver | 0.7 | 0.25-2 | (17, 18)(19) |
| K_{syn}_ALT | IU/cell/d | ALT synthesis by dHep | 8.1 × 10⁻⁸ | — | Derived⁸ |
| K_{syn}_DC | cell/mL/d | Naïve DC daily production | 1.076 × 10⁴ | — | Derived⁹ |
| K_{syn}_NK | cell/mL/d | Naïve NK daily production | 1.185 × 10⁵ | — | Derived¹⁰ |
| K_{prod antiHBs} | molec/cell/d | Antibodies against HBsAg daily production | 1.2 × 10⁹ | 1 × 10⁶-1.2 × 10⁹ | (17, 20, 21) |
| K_{prod antiHBc} | molec/cell/d | Antibodies against HBcAg daily production | 1.2 × 10⁹ | 1 × 10⁶-1.2 × 10⁹ | (17, 20, 21) |
| K_{syn CTL} | cell/mL/day | CTL production in the lymph node | 1000 | — | (7) |
| K_{rec}_CTL | cell/cell/day | CTL recruitment by DC in the lymph node | 0.1 | — | (7) |
| Kₐₛₜ_CTL | mL/cell/day | CTL activation by DC in the lymph node | 1 × 10⁻⁴ | 10⁻⁷-10⁻¹ | (7) |
| K_{syn}_Bcell | cell/mL/d | B cells daily production | 200 | — | Derived¹¹ |
| K_{act Bcell} | 1/d | Activation of B cells | 1 | — | (22) |

Degradation/Disappearance rate constants

| | | | | | |
|---|---|---|---|---|---|
| K_{death Hep} | 1/d | Natural death of healthy and infected hepatocytes | 0.0039 | 0.00693-0.0693 | (5,23) |
| K_{death}_dHep | 1/d | Disappearance of debris hepatocytes | 13 | — | (24)¹² |
| K_{deg HBV} | 1/d | Natural degradation of HBV | 0.7 | 0.67-1 | (4-6, 25) |
| K_{death DC} | 1/d | Natural death of activated and naive DCs | 0.231 | 0.23-0.35 | (26, 27) |
| K_{deg}_IFNα | 1/d | Degradation of IFNα | 5.74 | 2.16-9.78 | (27-29)¹³ |
| K_{death NK} | 1/d | Natural death of activated and naïve NKs | 0.069 | 0.013-0.111 | (23) |
| K_{deg IFNγ} | 1/d | Degradation of IFNγ | 33.4 | 28.51-39.92 | (30) |
| Kₑₓ_CTL* | 1/d | Exhaustion rate constant of CTL* | 0.403 | 1.21 × 10⁻⁸ − 6 | (31, 32)¹⁴ |
| K_{death}_CTL* | 1/d | Natural death of CTL* | 0.33 | 0.12-0.5 | (7, 33, 34) |
| K_{death}_CTLexh | 1/d | Natural death of exhausted CTL | 0.033 | — | Assumption¹⁵ |
| K_{death Treg} | 1/d | Natural death of Treg | 0.1 | 0.01-0.2 | (17, 35, 36) |
| K_{deg ALT} | 1/d | Degradation of ALT | 0.09 | — | Estimated |
| K_{deg HBsAg} | 1/d | Natural degradation of HBsAg | 0.0835 | 0.0835-0.122 | (37, 38) |
| K_{deg antiHBs} | 1/d | Natural degradation of antibodies against HBsAg | 0.033 | 0.033-0.125 | (20, 22, 39) |
| K_{deg complexHBV} | 1/d | Natural degradation of the complex HBV | 2.7 | | (39) |
| K_{deg}_complexantiHBs | 1/d | Natural degradation of the complex antiHBs | 2.7 | | (39) |
| K_{deg antiHBc} | 1/d | Natural degradation of antibodies against HBcAg | 0.033 | 0.033-0.125 | (20, 22, 39) |
| K_{death}_CTL | 1/d | Natural death of CTL in the lymph node | 0.102 | — | (7)¹⁶ |
| K_{death}_CTLm | 1/d | Natural death of memory CTL | 0.22 | — | Assumption¹⁷ |
| K_{death}_Bcell | 1/d | Natural death of naïve B cells | 0.2 | 0.2-2.4 | (17, 20) |
| MTT_{PB} | d | Mean transit time of plasmablasts | 50 | 2-50 | Calibrated |
| N_{PB} | unitless | Number of transit compartments of plasmablasts | 5 | 2-6 | (21) |
| K_{death PB} | 1/d | Natural death of plasmablasts | 0.1 | 0.05-0.1 | (21, 22) |
| K_{death PC} | 1/d | Natural death of plasma cells in plasma and lymph node | 0.1 | 0.05-1.2 | (17, 21, 22) |

Maximum concentration and ratio parameters

| | | | | | |
|---|---|---|---|---|---|
| Hepₜₒₜ | cell/mL | Total liver hepatocytes | 1.33 × 10⁸ | — | (6) |
| pDC_{RATIO} | unitless | Fraction of pDC from total DC* | 0.36 | 0.29-0.5 | (40) |
| CTL*_{RATIO} | unitless | Fraction of CTL* IFNγ+ | 0.65 | — | (14) |
| NK_{RATIO} | unitless | Ratio of TRAIL+ activated NK cells | 0.5 | 0.25-0.5 | (41) |
| CTL*ₘₐₓ_LV | cell/mL | Maximal concentration of specific CTL* in the liver | 2 × 10⁶ | 10⁶-10⁷ | (36) |
| Treg_{max LV} | cell/mL | Maximal concentration of Treg in the liver | 2 × 10⁶ | 10⁶-10⁷ | Calibrated |
| CTL*_{max LN} | cell/mL | Maximal concentration of specific CTL* in the lymph node | 105 | 3 × 10³ − 3 × 10⁵ | (7) |
| CTL_{m RATIO} | unitless | Fraction of CTL* proliferating as memory cells | 0.05 | 0.02-0.05 | (22, 42) |

Diffusion and binding rate constants

| | | | | | |
|---|---|---|---|---|---|
| K_{LV PL HBV} | 1/d | HBV distribution from liver to plasma | 1 | | Assumption |
| K_{PL LV HBV} | 1/d | HBV distribution from plasma to liver | 1 | | Assumption |
| K_{LV}_PL_HBsAg | 1/d | HBsAg distribution from liver to plasma | 1 | | Assumption |
| K_{PL}_LV_HBsAg | 1/d | HBsAg distribution from plasma to liver | 1 | | Assumption |
| K_{PL}_LV_DC | 1/d | DC distribution from plasma to liver | 0.0599 | | Derived¹⁸ |
| K_{LV}_LN_DC* | 1/d | DC* distribution from liver to lymph node | 0.9 | 0-1 | (17) |
| K_{PL}_LV_NK | 1/d | NK distribution from plasma to liver | 0.575 | | Derived¹⁹ |
| K_{PL}_LV_CTL* | 1/d | CTL* distribution from plasma to liver | 0.9 | 0.3-0.95 | Assumption²⁰ |
| K_{LV}_PL_ALT | 1/d | ALT distribution from liver to plasma | 0.09 | | Estimated |
| K_{LN}_PL_CTL* | 1/d | CTL * distribution from lymph node to plasma | 0.9 | 0.3-0.95 | (7) |
| K_{on antiHBs} | mL/molec/d | Association rate constant of HBV and HBsAg to antiHBs | 10⁻¹² | 10⁻⁹-10⁻¹² | (39) |
| K_{off antiHBs} | 1/d | Dissociation rate constant of complex antiHBs | 10 | — | (39) |
| K_{LN PL PC} | 1/d | PC distribution from lymph node to plasma | 0.9 | 0.3-0.95 | (17 |

Activity constants

| | | | | | |
|---|---|---|---|---|---|
| K_{lytic}_CTL | mL/cell/d | Cytopathic effect of CTL on iHep | 3.7 × 10⁻⁵ | 10⁻⁴-10⁻⁵ | (14)²¹ |
| K_{lytic NK} | mL/cell/d | Cytopathic effect of NK on iHep | 7.536 × 10⁻⁷ | (2.328-7.536) ×10⁻⁷ | (43) |
| IFNα₅₀_HBV | pg/mL | IFNα concentration inhibiting 50% of HBV synthesis by | 100 | 86.22-133.56 | (44, 45) |
| | | iHep | | | |
| IFNγ₅₀_HBV | pg/mL | IFNγ concentration inhibiting 50% of HBV synthesis by | 531.7 | 42-1020 | (46, 47)²² |
| | | iHep | | | |
| HBV₅₀_DC | virion/mL | HBV concentration triggering 50% of maximal DC | 10 | | Arbitrary |
| | | activation | | | |
| HBsAg_{LV}_50_IFNα | molecule/mL | HBsAg concentration in the liver inhibiting 50% IFNα | 2.20 × 10¹¹ | (1.28-3.10) × 10¹¹ | (48, 49)²³ |
| | | synthesis induced by DCa | | | |
| SLP_{IFNα}_NK | mL/pg | Fractional change on NK activation rate constant per IFNα | 5.25 | | (50, 51)²⁴ |
| | | unit change | | | |
| HBV₅₀_CTL_prol | virion/mL | HBV concentration triggering 50% of maximal CTL | 10 | | Arbitrary |
| | | proliferation | | | |
| γ_CTL | unitless | Shape parameter of HBV effect on CTL proliferation | 4 | | Arbitrary |
| Treg₅₀_CTL_exh | cell/mL | Treg concentration triggering 50% of maximal CTL | 10⁶ | — | Calibrated |
| | | exhaustion | | | |
| CTL₅₀_Treg_prol | cell/mL | CTL* concentration triggering 50% of maximal Treg | 10⁶ | — | Calibrated |
| | | proliferation in the liver | | | |
| DC₅₀_CTL_prol | cell/mL | DC concentration triggering 50% of maximal CTLa | 1 | | Arbitrary |
| | | proliferation in the lymph node | | | |
| DC₅₀_Bcell_prol | cell/mL | DC concentration triggering 50% of maximal B cell | 1 | | Arbitrary |
| | | proliferation in the lymph node | | | |

¹Calculated as the sum of the viral replication rate for both types of infected hepatocytes. 213 (± 157) + 102 (± 81) virions/(cell*day)
²Derived to achieve plasma levels ~ 50 μg/mL ranging from 30-300 μg/mL (37, 62, 63) assuming 85% of infected hepatocytes and a conversion factor of 3.011 × 10¹¹ molecules/μg
³Derived assuming a basal synthesis that maintain IFNα levels in healthy subjects. ksyn_{IFNα} = kdeg_{IFNα} * IFNα_{LV}
${}^{4} Derived to keep homeostasis : {kact}_{NK} = \frac{{k}_{death \_ NK} * {NK}_{LV}^{*}}{{NK}_{LV} * ( 1 + IFN {α}_{LV} * {SLP}_{IFN α - NK} )} = \frac{0.069 {d}^{- 1} * 3 e 5 cell / mL}{2.7 e 6 \frac{cell}{mL} * ( 1 + 80 \frac{pg}{mL} * 0.0035 \frac{mL}{pg} )}$ ${}^{5} Derived to keep homeostasis at basal level . {k}_{syn \_ IFN γ} = \frac{{k}_{deg \_ IFN γ} * IFN {γ}_{LV}}{{NK}_{LV}^{*}}$ ⁶Calculated assuming 2200 pg/mL/day is produced by the 65% of ca. 1e6 CTL cells.
⁷Logistic growth estimate corrected by CTL death term.
${}^{8} Derived assuming it comes from basal apoptotic hepatocytes : {k}_{syn \_ ALT} = \frac{( {k}_{LV \_ PL \_ ATL} + {k}_{deg \_ ALT} ) * {ALT}_{LV}}{dHep} = \frac{( 0.09 {d}^{- 1} + 0.09 {d}^{- 1} ) * 0.018 \frac{IU}{mL}}{40000 \frac{cell}{mL}}$ ${}^{9} Derived to keep homeostasis : {k}_{syn \_ DC} = ( {k}_{death \_ DC} + {k}_{PL - LV - D C} ) * D {C}_{PL} * {V}_{PL} = ( 0.231 {d}^{- 1} + 0.0599 {d}^{- 1} ) \times 37000 \frac{cell}{mL}$ ${}^{10} Derived to keep homeostasis : {k}_{syn \_ NK} = ( {k}_{death \_ NK} + {k}_{PL - LV - NK} ) * {NK}_{PL} = ( 0.069 {d}^{- 1} + 0.575 {d}^{- 1} ) * 1.8 e 5 \frac{cell}{mL}$ ${}^{11} Derived to keep homeostasis : {k}_{syn \_ Bcell} = {k}_{deg \_ Bcell} * Bcell ( 0 ) = 0.2 {d}^{- 1} * 1000 \frac{cell}{mL}$ ¹²Derived to keep homeostasis. Assuming that 2-4 cells per 10000 cells are apoptotic cells.
${k}_{death \_ dHep} = \frac{{k}_{death \_ Hep} * Hep ( 0 )}{dHep ( 0 )} = \frac{0.0039 {d}^{- 1} * 1.3 e 8 cells}{\frac{3}{10000} apoptotic cells * 1.3 e 8 cells}$ ¹³Derived assuming that IFNα half-life is 2.9 h.
¹⁴Reference Baral et al, refers to the maximal rate of exhaustion by antigen in hepatitis C.
¹⁵Assumed to be 10-fold lower than natural death of CTL*.
¹⁶Loss due to recirculation through the lymph node and death rate.
¹⁷Assumed to be 1/3 lower than natural death of CTL*.
${}^{18} Derived to keep homeostasis : {k}_{PL - LV - D C} = \frac{{k}_{death \_ D C} * D {C}_{LV} * {V}_{LV}}{D {C}_{PL} * {V}_{PL}} = \frac{0.231 {d}^{- 1} * 1.92 e 4 \frac{cell}{mL} * 1500 mL}{3.7 e 4 \frac{cell}{mL} * 3000 mL}$ ${}^{19} Derived to keep homeostasis : {k}_{PL - LV - NK} = \frac{( {k}_{death \_ NK} + {k}_{act \_ NK} ) * {NK}_{LV} * {V}_{LV}}{{NK}_{PL} * {V}_{PL}} = \frac{( 0.069 {d}^{- 1} + 0.00767 {d}^{- 1} ) * 2.7 e 6 \frac{cell}{mL} * 1500 mL}{1.8 e 5 \frac{cell}{mL} * 3000 mL}$ ²⁰Same value as for k_{LN}_PL_CTL*
²¹Estimated from in vitro experiments.
²²Estimated from in vitro experiments reported in those references.
²³Estimated from in vitro experiments reported in those references.
²⁴Estimated from in vitro experiments.

TABLE S2
III. Model Species

| Name | Compartment | Definition | Initial condition | Units | Ref. |
|---|---|---|---|---|---|
| Hep | Liver | Healthy hepatocytes | 1.33 × 10⁸ | cell/mL | (6, 52) |
| iHep | Liver | Infected hepatocytes | 0 | cell/mL | — |
| dHep | Liver | Debris death hepatocytes | 40000 | cell/mL | (24) |
| HBV_{LV} | Liver | HBV virions | 0.67 | virions/mL | (53) |
| HBsAg_{LV} | Liver | Hepatitis B surface antigen | 0 | molec/mL | — |
| DC_{LV} | Liver | Dendritic cells | 19200 | cell/mL | (54, 55)²⁵ |
| DC*_{LV} | Liver | Activated dendritic cell | 0 | cell/mL | — |
| IFNα_{LV} | Liver | Interferon alpha | 80 | pg/mL | Derived²⁶ |
| NK_{LV} | Liver | Natural killer | 2.7 × 10⁶ | cell/mL | (56, 57)²⁷ |
| NK*_{LV} | Liver | Activated natural killer | 3 × 10⁵ | cell/mL | (56, 57)²⁸ |
| NK^{TRAIL} | Liver | Activated natural killer cell TRAIL+ | 1.5 × 10⁵ | cell/mL | (41)²⁹ |
| IFNγ_{LV} | Liver | Interferon gamma | 30 | pg/mL | Derived³⁰ |
| CTL*_{LV} | Liver | HBV specific activated CTL | 0 | cell/mL | — |
| CTL^{IFNγ+}^{_{LV}} | Liver | IFNγ positive HBV specific activated CTL | 0 | cell/mL | — |
| CTLₑₓ | Liver | Exhausted CTL | 0 | cell/mL | — |
| Th0 | Liver | Regulatory T cells precursors | 2 × 10⁶ | cell/ml | (56, 58)³¹ |
| Treg | Liver | Regulatory T cells | 0 | cell/mL | |
| ALT_{LV} | Liver | Alanine aminotransferase | 0.018 | molec/mL | Derived³² |
| ALT_{PL} | Plasma | Alanine aminotransferase | 0.009 | molec/mL | (59) |
| HBsAg_{PL} | Plasma | Hepatitis B surface antigen | 0 | molec/mL | — |
| HBV_{PL} | Plasma | HBV virions | 0 | virions/mL | — |

²⁵Calculated assuming 0.23% of nonparenchymal cells (NPC) in the liver are DCs, and NPC represent 5% of total liver cells.
${}^{26} Calculated assuming proportionality between liver and plasma levels : IFN {α}_{LV} = IFN {α}_{PL} \times \frac{{V}_{PL}}{{V}_{LV}}$ ²⁷Assuming an average adult liver is likely to contain 10⁹-10¹⁰ lymphocytes (in 1500 mL), and that around the 30% of them are CD3-CD56+ cells and 90% of them inactive.
²⁸Assuming that in the liver, as in the blood, 10% of NK cells are activated.
²⁹Assuming 5% of active NK cells are TRAIL+.
³⁰Derived from IFNγ blood levels corrected by volumes.
³¹Assuming an average adult liver is likely to contain 10⁹-10¹⁰ lymphocytes (in 1500 mL), and around 60% are CD3+ and half of them are CD4+
${}^{32} Derived to keep homeostasis : {ALT}_{LV} ( 0 ) = \frac{{k}_{deg \_ ALT} \times {ALT}_{PL} \times {V}_{PL}}{{k}_{LV \_ PL \_ ALT} \times {V}_{LV}}$

### RECITATIONS OF ADDITIONAL EMBODIMENTS

Embodiment 1. A method for developing a quantitative system pharmacology (QSP) model to characterize one or more immune responses of an immune system against a hepatitis B virus (HBV) infection, the method comprising:
(a) building a QSP model comprising a plurality of parameters associated with the HBV infection;
(b) obtaining input values for the plurality of parameters for simulation;
(c) generating a plurality of predicted quantitative values of the immune responses across one or more anatomical compartments by applying the QSP model to the input values of the plurality of parameters for simulation;
(d) evaluating the QSP model by comparing the plurality of predicted quantitative values of the immune responses to observed data;
(e) assessing the QSP model by conducting an analysis; and
(f) analyzing behaviors predicted from the QSP model, wherein the immune responses of the immune system leads to one of self-resolution of an acute HBV infection, continuation of an acute status of the HBV infection, or evolution to a chronic status of the HBV infection.
Embodiment 2. The method of embodiment 1, wherein the plurality of parameters comprise one or both of CTL proliferation rate constant (kprol_CTL) and HBV synthesis constant (ksyn_HBV).
Embodiment 3. The method of embodiment 1 or 2, wherein the plurality of parameters comprise one or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.
Embodiment 4. The method of any one of embodiments 1-3, wherein the plurality of parameters comprise five or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.
Embodiment 5. The method of any one of embodiments 1-4, wherein the plurality of parameters comprise ten or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.
Embodiment 6. The method of any one of embodiments 1-5, wherein the plurality of parameters comprise each of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.
Embodiment 7. The method of any one of embodiments 1-6, wherein the plurality of parameters comprise at least one of a first set of parameters and a second set of parameters, wherein the first set of parameters reflects physiological conditions, and wherein the second set of parameters describes a rate of different biological and disease processes.
Embodiment 8. The method of any one of embodiments 1-7, wherein the one or more anatomical compartments comprise at least one of liver, plasma, and lymph node (LN).
Embodiment 9. The method of any one of embodiments 1-8, further comprising characterizing interaction between HBV and key components of immune systems across the one or more anatomical compartments.
Embodiment 10. The method of any one of embodiments 1-9, wherein the immune responses comprise at least one of innate immune response, adaptive immune response, and immunotolerant response, and wherein the adaptive immune response comprises at least one of HBV-specific cellular adaptive response and HBV-specific humoral response.
Embodiment 11. The method of any one of embodiments 1-10, wherein the immune system is initiated with a viral load arriving at a liver.
Embodiment 12. The method of any one of embodiments 1-11, wherein generating a plurality of predicted quantitative values of the immune responses across one or more anatomical compartments comprises: simulating dynamics of the plurality of parameters using software, wherein the software comprises the Simbiology® toolbox from Matlab® (R2019a).
Embodiment 13. The method of any one of embodiments 1-12, wherein building a QSP model comprises:
(i) providing initial conditions and initial parameters for model entities and parameter estimates;
(ii) implementing a plurality of biological entities across the one or more anatomical compartments; and
(iii) implementing a plurality of biological processes, the biological processes comprising at least one of synthesis, degradation, and distribution through at least one of zero-, first-, and second-order rate constants.
Embodiment 14. The method of any one of embodiments 1-13, wherein comparing the plurality of predicted quantitative values of the immune responses to observed data comprises at least one of:
(i) comparing a reproduction capability of the QSP model to general disease progression knowledge; and
(ii) comparing typical model predictions to clinical data.
Embodiment 15. The method of any one of embodiments 1-14, wherein assessing the QSP model by conducting an analysis comprises conducting at least one of a local sensitivity analysis or a parameter scan.
Embodiment 16. The method of any one of embodiments 1-16, wherein analyzing behaviors predicted from the QSP model comprise evaluating relative contribution of at least one of the immune responses on a time profile of at least one relevant disease biomarker.
Embodiment 17. The method of embodiment 16, wherein the at least one relevant disease biomarker comprises at least one of viral load, HBVs antigens, IFNα, and alanine aminotransferase (ALT).
Embodiment 18. The method of any one of embodiments 1-17, wherein analyzing behaviors predicted from the QSP model comprise evaluating a capability of the QSP model to predict development of chronicity of the immune system.
Embodiment 19. The method of any one of embodiments 1-18, wherein the QSP model is based on a topological network.
Embodiment 20. The method of embodiment 19, wherein the topological network comprises a proposed interaction between HBV and the immune responses across the one or more anatomical compartments.
Embodiment 21. The method of any one of embodiments 1-20, wherein the plurality of predicted quantitative values of the immune responses comprise time profiles for the plurality of parameters.
Embodiment 22. A non-transitory computer readable medium for developing a quantitative system pharmacology (QSP) model to characterize one or more immune responses of an immune system against a hepatitis B virus (HBV) infection, comprising instructions that, when executed by a processor, cause the processor to:
(a) build a QSP model comprising a plurality of parameters associated with the HBV infection;
(b) obtain input values for the plurality of parameters for simulation;
(c) generate a plurality of predicted quantitative values of the immune responses across one or more anatomical compartments by applying the QSP model to the input values of the plurality of parameters for simulation;
(d) evaluate the QSP model by comparing the plurality of predicted quantitative values of the immune responses to observed data;
(e) assess the QSP model by conducting an analysis; and
(f) analyze behaviors predicted from the QSP model, wherein the immune responses of the immune system leads to one of self-resolution of an acute HBV infection, continuation of an acute status of the HBV infection, or evolution to a chronic status of the HBV infection.
Embodiment 23. The non-transitory computer readable medium of embodiment 22, wherein the plurality of parameters comprise one or both of CTL proliferation rate constant (kprol_CTL) and HBV synthesis constant (ksyn_HBV).
Embodiment 24. The non-transitory computer readable medium of embodiment 22 or 4723 wherein the plurality of parameters comprise one or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.
Embodiment 25. The non-transitory computer readable medium of any one of embodiments 22-24, wherein the plurality of parameters comprise five or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.
Embodiment 26. The non-transitory computer readable medium of any one of embodiments 22-25, wherein the plurality of parameters comprise ten or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.
Embodiment 27. The non-transitory computer readable medium of any one of embodiments 22-26, wherein the plurality of parameters comprise each of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.
Embodiment 28. The non-transitory computer readable medium of any one of embodiments 22-27, wherein the plurality of parameters comprise at least one of a first set of parameters and a second set of parameters, wherein the first set of parameters reflects physiological conditions, and wherein the second set of parameters describes a rate of different biological and disease processes.
Embodiment 29. The non-transitory computer readable medium of any one of embodiments 22-28, wherein the one or more anatomical compartments comprise at least one of liver, plasma, and lymph node (LN).
Embodiment 30. The non-transitory computer readable medium of any one of embodiments 22-29, further comprising instructions that, when executed by the processor, cause the processor to characterize interaction between HBV and key components of immune systems across the one or more anatomical compartments.
Embodiment 31. The non-transitory computer readable medium of any one of embodiments 22-30, wherein the immune responses comprise at least one of innate immune response, adaptive immune response, and immunotolerant response, and wherein the adaptive immune response comprises at least one of HBV-specific cellular adaptive response and HBV-specific humoral response.
Embodiment 32. The non-transitory computer readable medium of any one of embodiments 22-31, wherein the immune system is initiated with a viral load arriving at a liver.
Embodiment 33. The non-transitory computer readable medium of any one of embodiments 22-32, wherein the instructions that cause the processor to generate a plurality of predicted quantitative values of the immune responses across one or more anatomical compartments comprises instructions that, when executed by the processor, cause the processor to: simulate dynamics of the plurality of parameters using software, wherein the software comprises the Simbiology® toolbox from Matlab® (R2019a).
Embodiment 34. The non-transitory computer readable medium of any one of embodiments 22-33, wherein the instructions that cause the processor to build a QSP model comprises instructions that, when executed by the processor, cause the processor to:
(i) provide initial conditions and initial parameters for model entities and parameter estimates;
(ii) implement a plurality of biological entities across the one or more anatomical compartments; and
(iii) implement a plurality of biological processes, the biological processes comprising at least one of synthesis, degradation, and distribution through at least one of zero-, first-, and second-order rate constants.
Embodiment 35. The non-transitory computer readable medium of any one of embodiments 22-34, wherein the instructions that cause the processor to compare the plurality of predicted quantitative values of the immune responses to observed data comprises instructions that, when executed by the processor, cause the processor to:
(i) compare a reproduction capability of the QSP model to general disease progression knowledge; or
(ii) compare typical model predictions to clinical data.
Embodiment 36. The non-transitory computer readable medium of any one of embodiments 22-35, wherein the instructions that cause the processor to assess the QSP model by conducting an analysis comprises instructions that, when executed by the processor, cause the processor to conduct at least one of a local sensitivity analysis or a parameter scan.
Embodiment 37. The non-transitory computer readable medium of any one of embodiments 22-36, wherein the instructions that cause the processor to analyze behaviors predicted from the QSP model comprise instructions that, when executed by the processor, cause the processor to evaluate relative contribution of at least one of the immune responses on a time profile of at least one relevant disease biomarker.
Embodiment 38. The non-transitory computer readable medium of embodiment 37, wherein the at least one relevant disease biomarker comprises at least one of viral load, HBVs antigens, IFNα, and alanine aminotransferase (ALT).
Embodiment 39. The non-transitory computer readable medium of any one of embodiments 22-38, wherein the instructions that cause the processor to analyze behaviors predicted from the QSP model comprise instructions that, when executed by the processor, cause the processor to evaluate a capability of the QSP model to predict development of chronicity of the c system.
Embodiment 40. The non-transitory computer readable medium of any one of embodiments 22-39, wherein the QSP model is based on a topological network.
Embodiment 41. The non-transitory computer readable medium of embodiment 40, wherein the topological network comprises a proposed interaction between HBV and the immune responses across the one or more anatomical compartments.
Embodiment 42. The non-transitory computer readable medium of any one of embodiments 22-41, wherein the plurality of predicted quantitative values of the immune responses comprise time profiles for the plurality of parameters.
Embodiment 43. A system for developing a quantitative system pharmacology (QSP) model to characterize one or more immune responses of an immune system against a hepatitis B virus (HBV) infection, comprising:
(a) a model building module configured to build a QSP model comprising a plurality of parameters associated with the HBV infection, wherein the model building module comprises:
(b) an input engine configured to obtain input values for the plurality of parameters for simulation;
(c) a prediction engine configured to generate a plurality of predicted quantitative values of the immune responses across one or more anatomical compartments by applying the QSP model to the input values of the plurality of parameters for simulation; and
(d) an evaluation engine configured to evaluate the QSP model by comparing the plurality of predicted quantitative values of the immune responses to observed data, assess the QSP model by conducting an analysis, and analyze behaviors predicted from the QSP model, wherein the immune responses of the immune system leads to one of self-resolution of an acute HBV infection, continuation of an acute status of the HBV infection, or evolution to a chronic status of the HBV infection.
Embodiment 44. The system of embodiment 43, wherein the plurality of parameters comprise one or both of CTL proliferation rate constant (kprol_CTL) and HBV synthesis constant (ksyn_HBV).
Embodiment 45. The system of embodiment 43 or 44, wherein the plurality of parameters comprise one or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.
Embodiment 46. The system of any one of embodiments 43-45, wherein the plurality of parameters comprise five or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.
Embodiment 47. The system of any one of embodiments 43-46, wherein the plurality of parameters comprise ten or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.
Embodiment 48. The system of any one of embodiments 43-47, wherein the plurality of parameters comprise each of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50_HBV, kact_DC, kdeg_NK, kp_LN_BL_DC, kp_CTL, kdeg_IFNa, or ksyn_HBsAg.
Embodiment 49. The system of any one of embodiments 43-48, wherein the plurality of parameters comprise at least one of a first set of parameters and a second set of parameters, wherein the first set of parameters reflects physiological conditions, and wherein the second set of parameters describes a rate of different biological and disease processes.
Embodiment 50. The system of any one of embodiments 43-49, wherein the one or more anatomical compartments comprise at least one of liver, plasma, and lymph node (LN).
Embodiment 51. The system of any one of embodiments 43-50, wherein the model building module further comprises a characterization engine configured to characterize interaction between HBV and key components of immune systems across the one or more anatomical compartments.
Embodiment 52. The system of any one of embodiments 43-51, wherein the immune responses comprise at least one of innate immune response, adaptive immune response, and immunotolerant response, and wherein the adaptive immune response comprises at least one of HBV-specific cellular adaptive response and HBV-specific humoral response.
Embodiment 53. The system of any one of embodiments 43-52, wherein the immune system is initiated with a viral load arriving at a liver.
Embodiment 54. The system of any one of embodiments 43-53, wherein generate a plurality of predicted quantitative values of the immune responses across one or more anatomical compartments comprises simulating dynamics of the plurality of parameters using software, wherein the software comprises the Simbiology® toolbox from Matlab® (R2019a).
Embodiment 55. The system of any one of embodiments 43-54, wherein build a QSP model comprises: providing initial conditions and initial parameters for model entities and parameter estimates to the model building module, wherein the model building module implements a plurality of biological entities across the one or more anatomical compartments, and wherein the model building module implements a plurality of biological processes, the biological processes comprising at least one of synthesis, degradation, and distribution through at least one of zero-, first-, and second-order rate constants.
Embodiment 56. The system of any one of embodiments 43-55, wherein comparing the plurality of predicted quantitative values of the immune responses to observed data comprises at least one of:
(i) comparing a reproduction capability of the QSP model to general disease progression knowledge using the evaluation engine; and
(ii) comparing typical model predictions to clinical data using the evaluation engine.
Embodiment 57. The system of any one of embodiments 43-56, wherein assess the QSP model by conducting an analysis comprises conducting at least one of a local sensitivity analysis or a parameter scan.
Embodiment 59. The system of any one of embodiments 43-57, wherein analyze behaviors predicted from the QSP model comprise evaluating relative contribution of at least one of the immune responses on a time profile of at least one relevant disease biomarker using the evaluation engine.
Embodiment 60. The system of embodiment 59, wherein the at least one relevant disease biomarker comprises at least one of viral load, HBVs antigens, IFNα, and alanine aminotransferase (ALT).
Embodiment 61. The system of any one of embodiments 43-60, wherein analyze behaviors predicted from the QSP model comprise evaluating a capability of the QSP model to predict development of chronicity of the immune system.
Embodiment 62. The system of any one of embodiments 43-61, wherein the QSP model is based on a topological network.
Embodiment 63. The system of embodiment 62, wherein the topological network comprises a proposed interaction between HBV and the immune responses across the one or more anatomical compartments.
Embodiment 64. The system of any one of embodiments 43-63, wherein the plurality of predicted quantitative values of the immune responses comprise time profiles for the plurality of parameters.

1. A method for characterizing immune responses of an immune system against a hepatitis B virus (HBV) infection, comprising:
(a) obtaining, by one or more processors, input values for a plurality of parameters associated with the HBV infection for simulation;
(b) generating a plurality of predicted quantitative values of the immune responses across one or more anatomical compartments by applying a quantitative system pharmacology (QSP) model to the input values of the plurality of parameters,
wherein the QSP model integrates immune response components involved in HBV viral clearance across the one or more anatomical compartments in a single framework, and
wherein the immune response components comprise innate immune response, adaptive immune response, and immunotolerant response; and
(c) characterizing the immune responses of the immune system based on the plurality of predicted quantitative values as leading to one of a self-resolution of an acute HBV infection, continuation of an acute status of the HBV infection, or evolution to a chronic status of the HBV infection.

2. The method of claim 1, wherein the plurality of parameters comprise one or both of CTL proliferation rate constant (kprol_CTL) and HBV synthesis constant (ksyn_HBV); one or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50 HBV, kact_DC, kdeg_NK, kp_LN_BL_DC, kp_CTL, kdeg_IFNa, or ksyn_HBsAg; and/or at least one of a first set of parameters and a second set of parameters, wherein the first set of parameters reflects physiological conditions, and wherein the second set of parameters describes a rate of different biological and disease processes.

3-7. (canceled)

8. The method of claim 1, wherein the one or more anatomical compartments comprise at least one of liver, plasma, bone marrow, lymphoid tissue, and lymph node (LN).

9. The method of claim 1, further comprising characterizing an interaction between HBV and the immune response components immune systems across the one or more anatomical compartments using the plurality of predicted quantitative values.

10. The method of claim 1, wherein the adaptive immune response comprises at least one of HBV-specific cellular adaptive response and HBV-specific humoral response.

11. The method of claim 1, wherein the immune system is initiated with a viral load arriving at liver.

12. The method of claim 1, wherein generating the plurality of predicted quantitative values comprises simulating dynamics of the plurality of parameters using software, wherein the software comprises the Simbiology® toolbox from Matlab® (R2019a).

13-33. (canceled)

34. A non-transitory computer readable medium for characterizing immune responses of an immune system against a hepatitis B virus (HBV) infection, comprising instructions that, when executed by a processor, cause the processor to:
(a) obtain input values for a plurality of parameters associated with the HBV infection for simulation;
(b) generate a plurality of predicted quantitative values of the immune responses across one or more anatomical compartments by applying a quantitative system pharmacology (QSP) model to the input values of the plurality of parameters,
wherein the QSP model integrates immune response components involved in HBV viral clearance across the one or more anatomical compartments in a single framework, and
wherein the immune response components comprise innate immune response, adaptive immune response, and immunotolerant response; and
(c) characterize the immune responses of the immune system based on the plurality of predicted quantitative values as leading to one of a self-resolution of an acute HBV infection, continuation of an acute status of the HBV infection, or evolution to a chronic status of the HBV infection.

35. The non-transitory computer readable medium of claim 34, wherein the plurality of parameters comprise one or both of CTL proliferation rate constant (kprol_CTL) and HBV synthesis constant (ksyn_HBV); one or more of kdeg_HBV, ksyn_HBV, Hep_tot, kinf, kdeath_CTL, IFNa50_HBV, kprof_CTL, kdeath_Treg, kp_HBV, kdeg_DC, MTT_PB, kexh_CTL, klytic_CTL, IFNg50 HBV, kact_DC, kdeg_NK, kp_LN_BL_DC, kp_CTL, kdeg_IFNa, or ksyn_HBsAg; and/or at least one of a first set of parameters and a second set of parameters, wherein the first set of parameters reflects physiological conditions, and wherein the second set of parameters describes a rate of different biological and disease processes.

36-40. (canceled)

41. The non-transitory computer readable medium of claim 34, wherein the one or more anatomical compartments comprise at least one of liver, plasma, bone marrow, lymphoid tissue, and lymph node (LN).

42. The non-transitory computer readable medium of claim 34, further comprising instructions that, when executed by the processor, cause the processor to characterize an interaction between HBV and the immune response components across the one or more anatomical compartments using the plurality of predicted quantitative values.

43. The non-transitory computer readable medium of claim 34, wherein the adaptive immune response comprises at least one of HBV-specific cellular adaptive response and HBV-specific humoral response.

44. The non-transitory computer readable medium of claim 34, wherein the immune system is initiated with a viral load arriving at liver.

45. The non-transitory computer readable medium of claim 34, wherein the instructions that cause the processor to generate the plurality of predicted quantitative values comprise instructions that, when executed by the processor, cause the processor to simulate dynamics of the plurality of parameters using software, wherein the software comprises the Simbiology® toolbox from Matlab® (R2019a).

46-66. (canceled)

67. A system for characterizing immune responses of an immune system against a hepatitis B virus (HBV) infection, comprising:
(a) a parameter value module configured to obtain input values for a plurality of parameters associated with the HBV infection for simulation;
(b) a module deployment module configured to generate a plurality of predicted quantitative values of the immune responses across one or more anatomical compartments by applying a quantitative system pharmacology (QSP) model to the input values of the plurality of parameters,
wherein the QSP model integrates immune response components involved in HBV viral clearance across the one or more anatomical compartments in a single framework, and
wherein the immune response components comprise innate immune response, adaptive immune response, and immunotolerant response; and
(c) a response characterization module configured to characterize the immune responses of the immune system based on the plurality of predicted quantitative values as leading to one of a self-resolution of an acute HBV infection, continuation of an acute status of the HBV infection, or evolution to a chronic status of the HBV infection.

68. The system of claim 67, wherein the plurality of parameters comprise one or both of CTL proliferation rate constant (kprol_CTL) and HBV synthesis constant (ksyn_HBV); one or more of kdeg_HBV, ksyn_HBV, Hep_tot, kinf, kdeath_CTL, IFNa50 HBV, kprof_CTL, kdeath_Treg, kp_HBV, kdeg_DC, MTT_PB, kexh_CTL, klytic_CTL, IFNg50 HBV, kact_DC, kdeg_NK, kp_LN_BL_DC, kp_CTL, kdeg_IFNa, or ksyn_HBsAg; and/or at least one of a first set of parameters and a second set of parameters, wherein the first set of parameters reflects physiological conditions, and wherein the second set of parameters describes a rate of different biological and disease processes.

69-73. (canceled)

74. The system of claim 67, wherein the one or more anatomical compartments comprise at least one of liver, plasma, bone marrow, lymphoid tissue, and lymph node (LN).

75. The system of claim 67, wherein the computer system further characterizes an interaction between HBV and the immune response components across the one or more anatomical compartments using the plurality of predicted quantitative values.

76. The system of claim 67, wherein the adaptive immune response comprises at least one of HBV-specific cellular adaptive response and HBV-specific humoral response.

77. The system of claim 67, wherein the immune system is initiated with a viral load arriving at liver.

78. The system of claim 67, wherein the prediction engine generates the plurality of predicted quantitative values by simulating dynamics of the plurality of parameters using software, wherein the software comprises the Simbiology® toolbox from Matlab® (R2019a).

79-99. (canceled)
